(19) United States
(12) Patent Application Publication
(10) Pub. No.: US 2010/0233135 A1
(43) Pub. Date: Sep. 16, 2010

(54) COMPOSITIONS AND METHODS FOR HEMATOPOIETIC STEM CELL EXPANSION OR FOR MODULATING ANGIOGENESIS

(75) Inventor: M. Amin Arnaout, Chestnut Hill, MA (US)

Correspondence Address: EDWARDS ANGELL PALMER & DODGE LLP, P.O. BOX 55874, BOSTON, MA, 02205, US

(73) Assignee: THE GENERAL HOSPITAL CORPORATION, Boston, MA (US)

(21) Appl. No.: 12/377,622

(22) PCT Filed: Aug. 16, 2007
(86) PCT No.: PCT/US07/18225

§ 371 Date: May 17, 2010

Related U.S. Application Data
(60) Provisional application No. 60/838,054, filed on Aug. 16, 2006.

(51) Int. Cl. A61K 35/14; C12N 5/078; A61K 38/17; A61P 9/10

(52) U.S. Cl. 424/93.7; 435/372; 514/12

(57) ABSTRACT

The present invention provides compositions and methods featuring ZBP-89 polypeptides or nucleic acid molecules for expanding a hematopoietic stem cell population or for modulating angiogenesis.

### CROSS-REFERENCE TO RELATED APPLICATIONS

This application claims the benefit of U.S. Provisional Patent Application No. 60/838,054, filed on Aug. 16, 2006, the entire contents of which are incorporated herein by reference.

### BACKGROUND OF THE INVENTION

Hematopoietic cell development is closely linked to the development of blood vessels and the two processes are regulated in large part by transcription factors that control cell fate decisions and cellular differentiation. Both blood and blood vessels derive from a common progenitor cell, termed the hemangioblast, but the factor(s) specifying development and differentiation of this stem cell population into the hematopoietic and vascular lineages remain ill defined. Methods for modulating the differentiation of this progenitor lineage are useful, for example, for increasing the numbers of hematopoietic cells. Hematopoietic stem cells, which give rise to cells of the blood and immune systems, may be used to restore or supplement a blood forming system or immune system compromised by radiation, chemotherapy, or disease. Methods for modulating blood vessel formation (e.g., angiogenesis or vasculogenesis) in a subject may be used to reduce angiogenesis related to tumor survival or growth, for example, or be used to increase angiogenesis in an ischemic condition. Because current methods are inadequate to modulate the development and differentiation of the hematopoietic and vascular lineages, improved therapeutic or prophylactic compositions and methods are urgently required.

### BRIEF SUMMARY OF THE INVENTION

The present invention provides compositions and methods for expanding a hematopoietic stem cell population or for modulating angiogenesis.

In one aspect, the invention generally provides a method for modulating (e.g., increasing or decreasing) hemangioblast cell fate. The method involves contacting a hemangioblast with an agent that alters expression of a ZBP-89 polypeptide or nucleic acid molecule in said hemangioblast, thereby modulating hemangioblast cell fate. the method increases or decreases ZBP-89 expression. In one embodiment, the method alters (e.g., increases or decreases) expression of Scl, Lmo2, or Gata-1 expression in the cell. In another embodiment, the cell assumes a hematopoietic or endothelial cell fate. In yet another embodiment, the method alters expression of flk1 or tie1.

In another aspect, the invention provides a method for increasing hematopoietic stem cell number, the method involving contacting a hematopoietic stem cell or progenitor cell with a ZBP-89 polypeptide, a nucleic acid encoding said polypeptide, or a mimetic thereof thereby increasing hematopoietic stem cell number.

In yet another aspect, the invention features a method for altering (e.g., increasing or decreasing) angiogenesis in a cell (e.g., a neoplastic cell), tissue or organ, the method involving contacting a cell, tissue, or organ with a ZBP-89 polypeptide, a nucleic acid encoding said polypeptide, a ZBP-89 inhibitory nucleic acid molecule, or a mimetic thereof, thereby altering angiogenesis.

In still another aspect, the invention features a method of treating a subject (e.g., a mammal, such as a human) in need of an increase in hematopoiesis. The method involves contacting a hematopoietic stem cell or progenitor cell of the subject with a ZBP-89 polypeptide or nucleic acid molecule; and increasing (e.g., by at least 5%, 10%, 25%, 50%, 75%, 95% or more) hematopoietic stem cell number, thereby treating the subject.

In still another aspect, the invention features method of providing hematopoietic cells in a subject in need thereof. The method involves administering a ZBP-89 polypeptide or nucleic acid molecule to the subject in an amount effective to increase hematopoietic stem or progenitor cell production in the subject, thereby providing hematopoietic cells to the subject.

In still another aspect, the invention features a method for modulating angiogenesis in a subject in need thereof. The method involves administering a therapeutically effective amount of a ZBP-89 polypeptide, nucleic acid molecule, or inhibitory nucleic acid molecule to the subject, thereby modulating angiogenesis in the subject.

In yet another aspect, the invention features an inhibitory nucleic acid molecule or functional mimetic thereof that is complementary to at least about 6 (e.g., 10, 12, 15, 20, 25, 30) nucleobases of a polynucleotide encoding a ZBP-89 polypeptide. In one embodiment, the nucleic acid molecule is an antisense oligonucleotide, an siRNA, or an shRNA molecule.

In a related aspect, the invention features an expression vector encoding the inhibitory nucleic acid molecule of the previous aspect.

In yet another aspect, the invention features a host cell (e.g., a human cell in vivo or in vitro) containing the expression vector of a previous aspect.

In still another aspect, the invention features a packaged pharmaceutical composition for increasing the number of hematopoietic stem cells, or progenitor cells in a subject, the composition containing a ZBP-89 polypeptide, or fragment thereof, in a pharmacologically acceptable excipient.

In yet another aspect, the invention features a packaged pharmaceutical composition for increasing the number of hematopoietic stem cells or progenitor cells in a subject, the composition containing a ZBP-89 nucleic acid molecule or fragment thereof, in a pharmacologically acceptable excipient.

In yet another aspect, the invention features a packaged pharmaceutical composition for inhibiting angiogenesis in a subject, the composition containing a ZBP-89 polypeptide, or fragment thereof, in a pharmacologically acceptable excipient.

In yet another aspect, the invention features a packaged pharmaceutical composition for inhibiting angiogenesis in a subject, the composition containing a ZBP-89 nucleic acid molecule, or fragment thereof, in a pharmacologically acceptable excipient.

In yet another aspect, the invention features a packaged pharmaceutical composition for increasing angiogenesis in a subject, the composition containing a ZBP-89 inhibitory nucleic acid molecule or a vector encoding said molecule, in a pharmacologically acceptable excipient.

In yet another aspect, the invention features a kit for increasing hematopoietic stem cell or progenitor cell number, the kit containing a ZBP-89 polypeptide, nucleic acid molecule, or fragment thereof.

In yet another aspect, the invention features a kit for altering angiogenesis in a subject, the kit containing a ZBP-89 polypeptide, nucleic acid molecule, or fragment thereof, or a ZBP-89 inhibitory nucleic acid molecule.

In various embodiments of the prior aspect, the kit further includes written instructions for the use of the kit.

In various embodiments of the previous aspects, the method further involves the step of obtaining the agent. In still other embodiments of any previous aspect, the ZBP-89 polypeptide, nucleic acid molecule, or fragment thereof, has at least about 85%, 90%, 95% or 99% sequence identity to a human ZBP-89 polypeptide. In still other embodiments of any previous aspect, the contacting occurs in vivo or in vitro. In still other embodiments of any previous aspect, the cell, hematopoietic stem cell, tissue, or organ is present in a subject (e.g., a human or veterinary patient). In still other embodiments of any previous aspect, the subject is a bone marrow donor or a transplant recipient, has anemia, or is identified as having a neoplasia (e.g., glioblastoma or renal cell carcinoma). In still other embodiments of any previous aspect, the subject is receiving chemotherapy, radiation therapy, or other cancer therapy. In still other embodiments of any previous aspect, the contacting increases or decreases expression of SCL, Lmo2, or Gata-1 and or the contacting increases or decreases expression of endothelial marker Flk1 or Tie1. In still other embodiments of any previous aspect, the ZBP-89 polypeptide, nucleic acid molecule, fragment or mimetic thereof is administered to the subject by an oral, intravenous, or transdermal route. In still other embodiments of any previous aspect, the ZBP-89 polypeptide is no longer expressed in the cells at the time of administering the cells to the subject. In still other embodiments of any previous aspect, the method further comprises administering the cells to a subject (e.g., oral, intravenous, or transdermal route). In still other embodiments of a previous aspect, the ZBP-89 polypeptide, nucleic acid molecule, or fragment thereof has at least about 80%, 85%, 90%, 95% or 99% sequence identity or complementarity to a reference sequence (e.g., a human ZBP-89 sequence). In still other embodiments of any previous aspect, the subject is in need of an increase or a decrease in angiogenesis. In still other embodiments of any previous aspect, contacting the cell with a ZBP-89 polypeptide or nucleic acid molecule reduces angiogenesis. In still other embodiments of any previous aspect, the subject has a condition characterized by an undesirable alteration in angiogenesis. In still other embodiments of any previous aspect, the subject has a condition that is a neoplasia, age-related macular degeneration, or diabetic retinopathy. In still other embodiments of any previous aspect, the contacting with a ZBP-89 inhibitory nucleic acid molecule increases angiogenesis. In still other embodiments of any previous aspect, the contacting reduces expression of scl, lmo2, or gata-1, or increases expression of flk1 or tie1. In still other embodiments of any previous aspect, the condition is an ischemic injury, myocardial infarction, or stroke.

Other features and advantages of the invention will be apparent from the detailed description, and from the claims.

### BRIEF DESCRIPTION OF THE FIGURES

FIGS. 1A and 1B show the organization of the ZBP-89 gene and its encoded protein. FIG. 1A is a schematic diagram that depicts the protein domains of ZBP-89. The genomic structure of human ZBP-89 is shown below, with the coding exons in yellow, non-coding exons in cyan, and introns represented by thick lines. The 24 kb intron 7 is interrupted in the figure. atgMO and spliceMO (indicated by red lines) represent the positions of the morpholino oligonucleotides used in the zebrafish knockdown experiments. FIG. 1B provides the amino acid sequence alignment for human and zebrafish ZBP-89. Identical amino acids are in red. The protein domains are boxed or overlined. Putative nuclear localization signals in the basic domains (BD) are overlined. Vertical blue lines indicate exon boundaries. The conserved cysteine-histidine (CH) linkers are underlined.

FIGS. 2A-2R are photomicrographs showing the expression profile of ZBP-89 in wild-type zebrafish embryos and the phenotype of ZBP-89 morphants. FIGS. 2A-2C show tissue expression of ZBP-89 by whole-mount in situ hybridization of 12 hours post fertilization (hpf) (2A), 18hpf (B), and 24 hpf (C) embryos. Lateral views, dorsal upward, anterior to the left are shown. By 12hpf, ZBP-89 is expressed in the anterior (arrowhead) and posterior (arrow) lateral plate mesoderm. At 18hpf (b), ZBP-89 is strongly expressed in the anterior intermediate cell mass (ICM) (arrow) and the brain region (arrowhead). At 24hpf, ZBP-89 expression in the anterior ICM (arrow) and wedge region of the anterior ICM (short arrow) are seen together with expression in brain (arrowhead). No signal was detected with the sense probe. FIGS. 2D-G show loss of ZBP-89 resulting in a bloodless phenotype via 2,7-diaminofluorene (DAF) staining of 48hpf whole mount zebrafish embryos. Blood (arrows) is present in representative control (mismatch atgMO)-(D,E) but not in atgMO-injected (F,G) embryos. Embryos are shown at 4× (D,E) and 10× (F,G) magnification. Different embryos are shown in FIGS. 2F and 2G. The bloodless phenotype was present in 78% and 62% of the 100-115 embryos injected at the 1-2 cell stage with either ZBP-89 atgMO or spliceMO, respectively. All views are lateral with anterior left and dorsal top. FIGS. 2H-2J depict rescue of ZBP-89 morphants by tissue-specific expression of human ZBP-89 under control of the flk1 promoter. Gata1-driven GFP in 22hpf transgenic (gata1:EGFP) zebrafish embryos are either untreated (FIG. 2H), injected with atgMO only (FIG. 2I), or atgMO plus flk1-ZBP-89 plasmid (FIG. 2J) at the 1-2 cell stage. Single 22hpf embryos were examined under a fluorescent microscope revealing gata1 expression (arrows) in the ICM of the untreated (FIG. 2H) and ZBP-89-rescued (FIG. 2J), but not in atgMO only-injected (FIG. 2I) embryos. FIGS. 2K-2R show the effect of ZBP-89 knockdown on expression of early hematopoiesis markers. Whole-mount in situ hybridization in wild type (WT) (FIGS. 2K, 2M, 2O, 2Q) and ZBP-89-depleted (FIGS. 2L, 2N, 2P, 2R) 24hpf embryos. Embryos were hybridized with digoxigenin-labeled RNA probes for scl (FIGS. 2K, 2L), lmo2 (FIGS. 2M, 2N) and gata-2 (FIGS. 2O, 2P). In WT, scl is expressed in the anterior ICM (FIG. 2K., arrow), posterior ICM (arrowhead in FIG. 2K), and the wedge region of anterior the ICM (FIG. 2K, short arrow). Only minimal expression remains in the wedge region and the posterior ICM in the ZBP-89 morphants (FIG. 2L). Lmo2 (FIG. 2M) and gata-2 (FIG. 2O) display a similar expression pattern to scl in WT embryos, and expression of both is markedly reduced in the ICM of ZBP-89 morphants (FIGS. 2N and 2P, respectively). Gata-2 is also expressed in brain and spinal ganglia (short arrows in FIGS. 2O, 2P); its expression at these sites is somewhat reduced by loss of ZBP-89. (FIGS. 2Q, 2R), expression of cdx4 in the posterior ICM in WT (FIG. 2Q) and ZBP-89 24hpf morphants (FIG. 2R). cdx4 expression is not affected by loss of ZBP.

FIGS. 3A-3T show expression of hematopoietic and vascular markers in WT and ZBP-89 morphants. FIGS. 3A-3R depict expression of primitive erythroid (A-D), primitive myeloid (E-J), and definitive hematopoietic markers (K-R). Normal expression (arrows) of the primitive erythroid genes gata-1 (A) and tif1g (C) in the anterior ICM of WT embryos is almost completely lost in ZBP-89 morphants (B, D, respectively). Expression of tif1g in neural tissue (arrowheads) is not affected. Expression of the primitive myeloid markers pu.1, mpo, and l-plastin in WT (E, G and I, respectively) and in ZBP-89 morphants (F, H, J, respectively). The normal expression of pu.1 in primitive macrophages in the anterior ICM (arrow) in 24hpf embryos (E) is markedly reduced by depletion of ZBP-89 (F). Its expression is also reduced in 20hpf embryos in the head, rostral blood islands and ICM (data not shown). mpo (G) and l-plastin (I) are normally expressed in the ICM of 24hpf embryos (arrows) and in the anterior yolk region (l-plastin). Both markers are severely reduced by loss of ZBP-89 (H and J, respectively). (K-R) Expression of the definitive hematopoietic markers runx1 and c-myb. Expression of runx1 begins in the ICM (K, arrows) at 24 hpf and is well developed in the ventral dorsal aorta at 48hpf (M, arrows). Loss of ZBP-89 markedly reduces expression of runx1 in 24hpf (L) and 48hpf (N) embryos. c-myb is normally expressed in the ICM of WT embryos at 24hpf (O, arrow). In 48hpf embryos, cells expressing c-myb are found scattered along the ventral wall of the dorsal aorta (Q, arrows), within the first progenitors of definitive hematopoiesis. c-myb expression is significantly reduced in ZBP-89 morphants in both 24- and 48hpf embryos (P and R, respectively). Non-hematopoietic expression of runx1 (K, L) and c-myb (0, P) in neural tissue (arrowheads) was not affected by loss of ZBP-89. (S, T) flk1 expression in 20 hpf WT and ZBP-89 morphants. flk1 is normally expressed in cells located in two strips of the anterior lateral mesoderm (short arrows), and in the forming anterior (arrows), and posterior (arrowheads) ICM (S). This expression was not affected by loss of ZBP-89 (1). All views are lateral with anterior left and dorsal top.

FIGS. 4A-4L show that ZBP-89 rescues the hematopoietic but not the vascular phenotype in clo^{-/-} mutant zebrafish embryos. FIGS. 4A-4B show WT whole mount 3 days postfertilization (dpf) embryos stained with DAF and examined at 4× (A) and 10× (B) magnification, showing blood cells in the heart (arrow), the dorsal aorta (arrow, B), and posterior cardinal vein (arrowhead, B). FIGS. 4C-D show Clo^{-/-} embryo at 4× (C) and 10× (D) magnification. No extravascular or circulating blood is seen. The arrow in FIG. 4C indicates the dilated bloodless heart. FIGS. 4E-4F: ZBP-89 sense RNA was injected into clo^{-/-} embryos shown at 4× (E) and 10× (F) magnification. Blood formation in the trunk is evident (arrow in 4F), but the heart chamber remains dilated and devoid of erythrocytes, consistent with the absence of blood vessels. FIGS. 4G, 4H:Gata1-driven GFP in clo^{-/-} 48 hpf embryos transgenic (Tg) for gata-1:EGFP before (G) and after (H) overexpression of ZBP-89. Gata1 is found in the GFP-labeled erythroid lineage (arrow in H) in the anterior ICM of clo^{-/-} embryos overexpressing ZBP-89. FIGS. 4I-K show transgenic (Flk1:EGFP) 48hpf embryos showing fluorescence in the axial (arrow in I) and intersomitic (short arrow in I) blood vessels; fluorescence in the yolk extension is nonspecific. FIGS. 4J and 5K show Flk1-driven GFP in clo^{-/-} 48hpf embryos transgenic (Tg) for flk1:EGFP before (4J) and after (4K) overexpression of ZBP-89. Arrows show lack of blood vessel formation in both cases. FIG. 4L is an agarose gel showing the ZBP-89 expression profile in two different wild type (WT1 and WT2) and clo^{-/-} 18hpf embryos (clo^{-/-} 1 and clo^{-/-} 2) assessed using RT-PCR. GAPDH expression was examined simultaneously as a reference.

FIGS. 5A-5F shows rescue of ZBP-89 morphants by zebrafish scl sense RNA. Gata1-driven GFP in 22hpf transgenic (gata1:EGFP) zebrafish embryos are either untreated (5A, 5D), injected with atgMO only (5B, 5E) or atgMO plus scl RNA (5C, 5F) at the 1-2 cell stage. FIGS. 5A-C show single 22hpf embryos examined under a fluorescent microscope revealing gata1 expression (red arrows) in the ICM of the untreated (5A) and sd-rescued (5C), but not in atgMO only-injected (5B) embryos. FIGS. 5E-5G are bright-field low magnification images of the respective untreated and treated Tg gata1:EGFP embryos at 48hpf. Short arrows in (5E) point to pericardial edema in the ZBP-89 morphants.

FIGS. 6A-6L show the effect of forced expression of ZBP-89 on hematopoietic and vascular development in zebrafish embryos. WT (A.C.E.G.I) and ZBP-89-overexpressing (B,D,F,H,J) 18hpf (A-F) or 24hpf (G-J) zebrafish embryos. In situ hybridization of WT embryos overexpressing ZBP-89 reveals a marked increase in expression of scl (B), lmo2 (D), and gata-1 (F), but a marked reduction in intersomitic expression of the endothelial markers flk1 (H) and tie1 (J), when compared to the respective untreated WT embryos (A,C,E,G,I). WT (K) and ZBP-89-overexpressing (L) Tg (flk1:EGFP) zebrafish embryos examined at 24hpf. Overexpression of ZBP-89 caused a significant reduction in flk1 expression in the axial (arrows), brain (short arrow), and intersomitic (arrow heads) blood vessels, when compared to the control embryo.

FIGS. 7A-7D show the expression profile of ZBP-89 and effects of its overexpression on hematopoiesis in mouse embryoid body (EB) cultures. FIG. 7A provides a ZBP-89 expression profile in undifferentiated embryonic stem cells (ESCs) and differentiating EBs quantified with real-time PCR. Numbers indicate day of differentiation. Results represent mean±SD of 3 independent experiments. Inset is a Western blot analysis showing induction of the ZBP-89 protein mainly in FLK1⁺ mesoderm precursors in day-3 embryoid bodies. Lane 1, positive control; lane 2, uninduced ESCs; lane 3, day-3 FLK1⁻ mesodermal cells; lane 4 FLK1⁺ mesoderm precursors. Equivalent amounts of cell lysate were loaded per lane as reflected by the β-actin signal. FIG. 7B provides histograms showing number of blast colony-forming cells (BL-CFCs), secondary embryoid bodies (2° EBs), and primitive erythroid (Erythroid burst-forming units (BFU-Es)) colonies generated from control, D5 and E1 clones (bars represent the mean number of colonies+SD from two independent experiments). FIG. 7C provides histograms (mean±SD, n=3) showing the numbers of definitive erythroid (CFU-E), macrophage (CFU-M), granulocyte-macrophage-megakaryocyte (CFU-GEMM), and granulocyte-macrophage (CFU-GM) colonies. * and ** indicate p<0.01 and p<0.001, respectively (paired t test). FIG. 7D shows flow cytometric analysis of single cell suspension from E1-derived embryoid bodies stained with FITC-labeled rat anti-mouse CD45 monoclonal antibody. ZBP-89 overexpression significantly increased the number of CD45⁺ hematopoietic progenitors.

FIGS. 8A-8D show that ectopic expression of ZBP-89 impairs angiogenesis in mouse EB cultures. FIG. 8A is a graph. Primary day-11 embryoid bodies were dissociated and cultured in collagen matrix with growth factors for endothelial cell differentiation for four days, Histograms (mean±SD, n=3) showing the number of primary day-11 embryoid bodies with 0, <5 or >5 branching vessels/embryoid body derived from the D5 and E1 clones compared to the control. The following symbols: “*,” “**” and “NS” denote <0.01, p<0.001 and not significant, respectively. FIG. 8B provides representative images of control-, E1-, or D5-derived sprouting embryoid body colonies. The reduction in the branching vessels (arrows in controls) in embryoid bodies derived from overexpressing ZBP-89 clones E1 and D5 is also clearly reflected by the compactness of the respective colonies as a result of the fewer branches produced compared to controls. FIGS. 8C, and 8D show flow cytometric analyses of single cell suspension of embryoid bodies overexpressing ZBP-89 stained with phycoerythrin (PE)-labeled anti-CD31 (day 6 culture) or anti-vascular endothelial (VE)-Cadherin (day 7 and day 10 cultures) antibodies. ZBP-89 overexpression significantly reduced the number of CD31⁺ and VE-Cadherin⁺ angioblasts (with similar results obtained in two other independent experiments).

FIG. 9 shows expression of ZBP-89 in early hematopoietic progenitors and in angioblasts in day 4 embryoid body cultures. The plot depicted results from FACS sorting carried out using FLK1 and Stem Cell Leukemia (SCL) (human CD4) markers. The inset is an agarose gel showing the results of semi-quantitative RT-PCR of the ZBP-89 expression (white arrow) profile in hemangioblasts FLK1⁺SCL⁺ (F+/S+), angioblasts FLK1⁺SCL⁻ (F+S−) and hematopoietic progenitors FLK1⁻SCL⁺ (F−S+), as defined by the expression of FLK1 (F) and SCL (S). β-actin expression (black arrow) was used as internal control. “M” denotes markers. “H2O” is the negative control lane.

FIGS. 10A and 10B show the results of PCR analysis and flow cytometry of Day 4 embryoid bodies generated from embryonic stem cells that are WT (ZBP-89^{+/+}), heterozygous for a foxed ZBP-89 allele (ZBP-89^{Flox/+})(clone #6071), for the LacZ allele ZBP-89^{lacZ/+}, or clones H2, H3 and H5, in which the WT allele in ZBP-89^{lacZ+} has been knocked down by siRNAs. FIG. 10A is an agarose gel showing the results of a semi quantitative RT-PCR assessing expression of ZBP-89 and β-actin in WT- and ZBP-89 mutant EBs at day 4. Mr, molecular mass markers. FIG. 10B is a FACS analysis of the derived embryoid bodies immunostained with, anti-FLK1 monoclonal antibody (mAb).

FIGS. 11A-11C show that the loss of ZBP-89 impairs hematopoietic development in mouse embryoid body cultures. FIG. 11A is a histogram showing the number of BL-CFCs, 2° Ebs, and BFU-Es colonies generated from control (WT), E1, and H5 ESC clones (mean±SD, n=3). ** p<0.001 (paired t test). FIG. 11B is a histogram (mean±SD, n=3) showing the numbers of definitive erythroid (CFU-E), macrophage (CFU-M), granulocyte-macrophage-megakaryocyte (CFU-GEMM), and granulocyte-macrophage (CFU-GM) colonies from WT, (control), E1 (overexpressing ZBP-89), and H5 (ZBP-89 knocked down). FIG. 11C shows plots resulting from a FACS analysis of SCA-1⁺/c-KIT⁺ hematopoietic stem cell population in day 3 embryoid bodies derived from the respective embryonic stem cells. This experiment is a representative of three.

FIG. 12 shows that stable knockdown of ZBP-89 enhances sprouting angiogenesis in mouse embryoid body cultures. FIG. 12A is a histogram (mean±SD, n=3) showing the number of primary day-11 embryoid bodies with 0, <5 or >5 branching, vessels/embryoid body derived from the WT (control), E1 (ectopic expression), and H5 (siRNA knockdown) clones. The symbol “**” indicates p<0.001. The inset shows a representative photomicrograph of WT (control)- or H5-derived sprouting embryoid body colonies. The branching vessels in control embryoid bodies (arrowheads) are massively increased in embryoid bodies derived from H5 (the EB colony has lost its spherical architecture into massive number of vessels visualized under phase microscopy).

FIGS. 13A-E depict conditional targeting of ZBP-89. FIG. 13A schematically depicts the recombinant locus. Exon 8 and exon 9 are drawn to scale. Oligonucleotides used in PCR reactions are indicated by thick short vertical lines (Oligos 3392For and 3′IKO Flank are outside the recombined segment; oligos in black have WT sequence; oligos in gray contain Neo-resistance gene (NEO) sequence and 10631F oligo (in dark gray) includes the 3′ loxP sequence). FIGS. 13B and 13C are agarose gels showing products of PCR reactions conducted on embryonic stem cell (ESC)-(13B, 13C) or mouse tail-(C) derived genomic DNA from NEO and WT ESC clones, as well as two male (m) and two female (F) mice. Flanking 5′ (3.2 kb) and 3′ (5.6 kb) PCR products of the expected size for the floxed ZBP-89 allele were obtained from all five (but not the WT) ESCs and from two (257m and 258F) adult mice confirming germline transmission of the foxed ZBP-89 allele. FIG. 131) are agarose gels showing PCR products from tail genomic DNA or derived from ESCs 6071 and 6072 and from ZBP-89^{flox/+}, using forward and reverse oligos if-2r and 3f-4-r (on either side of the 3′LoxP sequence). Using these two sets, ZBP-89^{flox+} ESCs and floxed mice produced the expected foxed (0.4 kb) band (upper panel) and WT (200 bp) and floxed (233 bp) bands (lower panel). The term “m” denotes male and “F” denotes female. FIG. 13E is an agarose gel showing proper excision of the exon8-Neo-exon 9 cassette by transient introduction of Tet-ON-regulated Cre recombinase into ESC clone 6071. The primers used are the same as in (D). Expression of Cre by addition of tetracycline (6071+Cre) led to loss of the 0.4 kb and 233 bp bands corresponding to the foxed allele.

FIG. 14 lists peripheral blood counts from the heparinized blood of a heterozygous (upper panel) and two homozygous (lower panels) mice. Note the dramatic reduction in red blood cells (RBC) and platelets in ZBP-89^{flox/flox} mice and the moderate reduction in these two parameters in the heterozygous mouse.

FIG. 15 schematically depicts the potential sites of action of ZBP-89.

### DEFINITIONS

By “ZBP-89 polypeptide” is meant a protein having at least 85% amino acid identity to ZBP-89 polypeptide, or a functional fragment or functional mimetic thereof, that alters the survival or proliferation of a hematopoietic stem cell or that alters angiogenesis. One exemplary ZBP-89 polypeptide is provided at NCBI Accession No. AAC39926. By “ZBP-89 biological activity” is meant any activity that alters the survival or proliferation of a hematopoietic stem cell or stem cell progenitor or that alters angiogenesis.

By “ZBP-89 nucleic acid molecule” is meant a polynucleotide that encodes an ZBP-89 polypeptide as defined above. One exemplary ZBP-89 nucleic acid molecule is provided at GenBank Accession No. AF039019.

By “agent” is meant any small molecule chemical compound, antibody, nucleic acid molecule, or polypeptide, or fragments thereof.

By “ameliorate” is meant decrease, suppress, attenuate, diminish, arrest, or stabilize the development or progression of a disease.

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and more preferably a 50%, 60%, 75%, 85%, 95% or greater change in expression levels.

By “cell fate” is meant the differentiated state of the cell.

In this disclosure, “comprises,” “comprising,” “containing” and “having” and the like can have the meaning ascribed to them in U.S. Patent law and can mean “includes,” “including,” and the like; “consisting essentially of or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

By “condition” is meant any disease or disorder that damages or interferes with the normal function of a cell, tissue, or organ.

“Expansion” refers to the propagation of a cell or cells without terminal differentiation.

“Differentiation” refers to the developmental process of lineage commitment. A “lineage” refers to a pathway of cellular development, in which precursor or “progenitor” cells undergo progressive physiological changes to become a specified cell type having a characteristic function (e.g., nerve cell, muscle cell or endothelial cell). Differentiation occurs in stages, whereby cells gradually become more specified until they reach full maturity, which is also referred to as “terminal differentiation.” A “terminally differentiated cell” is a cell that has committed to a specific lineage, and has reached the end stage of differentiation (i.e., a cell that has fully matured).

By “an effective amount” is meant the amount of a required to ameliorate the symptoms of a disease relative to an untreated patient. The effective amount of active compound(s) used to practice the present invention for therapeutic treatment of a neurodegenerative disease varies depending upon the manner of administration, the age, body weight, and general health of the subject. Ultimately, the attending physician or veterinarian will decide the appropriate amount and dosage regimen. Such amount is referred to as an “effective” amount.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

By “hybridize” is meant pair to form a double-stranded molecule between complementary polynucleotide sequences.

By “inhibitory nucleic acid” is meant a double-stranded RNA, siRNA, shRNA, or antisense RNA, or a portion thereof, or a mimetic thereof, that when administered to a cell results in a decrease (e.g., by 10%, 25%, 50%, 75%, or even 90-100%) in the expression of a target gene.

By “marker” is meant any protein or polynucleotide having an alteration in expression level or activity that is associated with a condition, disease or disorder.

By “obtain” is meant purchasing, synthesizing, or otherwise acquiring.

By “nucleic acid” is meant an oligomer or polymer of ribonucleic acid or deoxyribonucleic acid, or analog thereof. This term includes oligomers consisting of naturally occurring bases, sugars, and intersugar (backbone) linkages as well as oligomers having non-naturally occurring portions which function similarly. Such modified or substituted oligonucleotides are often preferred over native forms because of properties such as, for example, enhanced cellular uptake and increased stability in the presence of nucleases.

By “reference” is meant a standard or control condition.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or there between.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency (e.g., stringent conditions). (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and more preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100.mu.g/ml denatured salmon sperm DNA (ssDNA). In a more preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and more preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42.degree. C. in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is between 80%-99% identical (e.g., where the bottom of the range is any integer between 80 and 98%, and the top of the range is any integer between 81% and 99%). In one embodiment, the sequence is at least about 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “subject” is meant a mammal, including, but not limited to, a human or non-human mammal, such as a bovine, equine, canine, ovine, or feline.

The term “stem cell” is meant a multipotent or pluripotent cell having the capacity to self-renew and to differentiate into multiple cell lineages.

By “stem cell generation” is meant any biological process that gives rise to stem cells. Such processes include the differentiation or proliferation of a stem cell progenitor or stem cell self-renewal.

By “stem cell progenitor” is meant a cell that gives rise to stem cells.

### DETAILED DESCRIPTION OF THE INVENTION

The present invention provides compositions and methods for modulating hematopoiesis and angiogenesis that feature ZBP-89 polypeptide and nucleic acid molecules. The invention is based, at least in part, on the observation that ZBP-89 is a lineage determining transcription factor that not only activates hematopoietic lineage-specific genetic programs, but that also suppresses endothelial cell differentiation. As reported herein, ZBP-89 functions in embryonic blood and endothelial cell development. ZBP-89 was found to play a central role in hemangioblast fate determination by inducing expression of SCL, which triggers HSC differentiation, and by suppressing angioblast differentiation. These opposing effects indicate that ZBP-89 may be used to increase numbers of HSC (e.g., in BM transplantation) or as a pro- or anti-angiogenic drug.

### Hematopoietic Development

Hematopoietic development is closely linked to the development of blood vessels. Vertebrate hematopoiesis occurs in two developmental waves: a short primitive wave predominantly generating erythrocytes and primitive myeloid cells, and a definitive wave producing long-term hematopoietic stem cells. Hematopoietic stem cell (HSCs) progenitors are believed to arise from bipotential fetal liver kinase-1⁺ (FLK1⁺) mesoderm stem cells, which also give rise to vascular progenitors. The genetic regulatory networks that control blood and blood vessel development are regulated in large part by transcription factors that control cell fate decisions and cellular differentiation. One such factor is the product of the cloche (clo) gene, which is needed for generating both the hematopoietic and vascular progenitors, but its nature remains to be defined. A second factor, SCL is a basic helix-loop-helix transcription factor encoded by the scl/tal-1 gene that has been shown to be important in directing hematopoietic fate commitment from hemangioblasts, as well as in embryonic angiogenesis.

The hematopoietic and endothelial lineages can be produced in vitro from murine embryonic stem cell (ESC)-derived embryoid bodies (EB), in a temporal pattern that recapitulates the development of these cell populations in vivo (Palis et al., 1999). Analysis of early EBs, between days 2.5 and 4 of ESC differentiation prior to hematopoietic and endothelial lineage commitment, reveals the presence of a transient mesoderm-derived FLK1⁺SCL⁺ progenitor, or blast colony-forming cell (BL-CFC), which represents the in vitro equivalent of the yolk sac hemangioblast (Chung et al., 2002; D'Souza et al., 2005; Fehling et al., 2003; Park et al., 2005). Expression of c-kit in this population marks a hematopoietic potential (Willey et al., 2006). Both blood and blood vessels derive from a common progenitor, termed the hemangioblast, but the factor(s) specifying development and differentiation of this stem cell population into the hematopoietic and vascular lineages remain ill defined. As reported in more detail below, knockdown of the Krüppel-like transcription factor ZBP-89 in zebrafish embryos resulted in a bloodless phenotype, caused by disruption of both primitive and definitive hematopoiesis, while leaving primary blood vessel formation intact.

### ZBP-89

ZBP-89 (ZNF148) is the prototype of a novel class of transcription factors, phylogenetically conserved in mammals, that contains a characteristic array of three N-terminal C₂H₂ Krüppel-like zinc-fingers and a forth C2HC variant zinc-finger. It shares with members of the Krüppel-like finger (KLF) protein family with three Krüppel-like zinc-fingers (Bray et al., 1991); ZBP-89 has a forth zinc-finger and all four are located in the N-terminal region in contrast to the conserved C-terminal location of the zinc-fingers in the KLF protein family (Kaczynski et al., 2003). The ZBP-89 gene is localized on chromosome 3q21, the site of breakpoints (Pekarsky et al., 1995) and translocations (Yamagata et al., 1997) in some cases of acute myeloid leukemia (Antona et al., 1998; Bernstein et al., 1986), but it is not clear if the any involve the ZBP89 gene itself. In the only in vivo study to date, haploinsufficiency of ZBP-89 caused infertility in normally developed male mice due to growth arrest and apoptosis of fetal germ cells (Takeuchi et al., 2003). It was shown that ZBP-89 represses expression of the myeloid differentiation marker CD11b in vitro (Park et al., 2003). To determine whether ZBP-89 functions in hematopoiesis in vivo, the zebrafish ortholog of ZBP-89 was cloned, its expression was analyzed, and the phenotype that resulted from modulating ZBP-89 expression in zebrafish embryos and murine EB cultures was characterized.

Injection of ZBP-89 mRNA into cloche zebrafish embryos, which lack both the hematopoietic and endothelial lineages, rescued hematopoiesis but not vasculogenesis. Injection of mRNA for Stem Cell Leukemia (SCL), a transcription factor that directs hemangioblast development into blood cell precursors, rescued the bloodless phenotype in ZBP-89 zebrafish morphants. Forced expression of ZBP-89 induced the expansion of hematopoietic progenitors in wild-type zebrafish and in mouse embryonic stem cell cultures, but inhibited angiogenesis in vivo and in vitro. These findings established a unique regulatory role for ZBP-89, positioned at the interface between early blood and blood vessel development. Accordingly, compositions and methods that increase ZBP-89 levels may be used to increase the number of HSC in vitro or in vivo. Such methods are useful in treating subjects in need of increased HSC numbers, for example, subjects having BM transplantation. Results showing that increased expression of ZBP-89 suppressed angiogenesis indicating that ZBP-89 is also likely to be useful as anti-angiogenic drug.

### Treatment Methods Related to Stem Cell Expansion.

In view of the results described herein, ZBP-89 may be used to increase the number of hematopoietic stem cells in a subject. Accordingly, the methods of the invention can be used to treat a disease or disorder in which it is desirable to increase the number of hematopoietic stem cells or their progenitors. Frequently, subjects in need of the inventive treatment methods will be those undergoing or expecting to undergo an immune cell depleting treatment, such as chemotherapy. Most chemotherapy agents used act by killing all cells going through cell division. Bone marrow is one of the most prolific tissues in the body and is therefore often the organ that is initially damaged by chemotherapy drugs. The result is that blood cell production is rapidly destroyed during chemotherapy treatment, and chemotherapy must be terminated to allow the hematopoietic system to replenish the blood cell supplies before a patient is re-treated with chemotherapy.

Thus, methods of the invention can be used, for example, to treat patients requiring a bone marrow transplant or a hematopoietic stem cell transplant, such as cancer patients undergoing chemo and/or radiation therapy. Methods of the present invention are particularly useful in the treatment of patients undergoing chemotherapy or radiation therapy for cancer, including patients suffering from myeloma, non-Hodgkin's lymphoma, Hodgkins lymphoma, or leukaemia.

Disorders treated by methods of the invention can be the result of an undesired side effect or complication of another primary treatment, such as radiation therapy, chemotherapy, or treatment with a bone marrow suppressive drug, such as zidovadine, chloramphenical or gangciclovir. Such disorders include neutropenias, anemias, thrombocytopenia, and immune dysfunction. In addition, methods of the invention can be used to treat damage to the bone marrow caused by unintentional exposure to toxic agents or radiation.

Methods of the invention can further be used as a means to increase the number of mature cells derived from hematopoietic stem cells (e.g., erythrocytes). For example, disorders or diseases characterized by a lack of blood cells, or a defect in blood cells, can be treated by increasing the pool of hematopoietic stem cells. Such conditions include thrombocytopenia (platelet deficiency), and anemias such as aplastic anemia, sickle cell anemia, Fanconi's anemia, and acute lymphocytic anemia. In addition to the above, further conditions which can benefit from treatment using methods of the invention include, but are not limited to, lymphocytopenia, lymphorrhea, lymphostasis, erythrocytopenia, erthrodegenerative disorders, erythroblastopenia, leukoerythroblastosis; erythroclasis, thalassemia, myelofibrosis, thrombocytopenia, disseminated intravascular coagulation (DIC), immune (autoimmune) thrombocytopenic purpura (ITP), HIV inducted ITP, myelodysplasia; thrombocytotic disease, thrombocytosis, congenital neutropenias (such as Kostmann's syndrome and Schwachman-Diamond syndrome), neoplastic associated-neutropenias, childhood and adult cyclic neutropaenia; post-infective neutropaenia; myelo-dysplastic syndrome; and neutropaenia associated with chemotherapy and radiotherapy.

The disorder to be treated can also be the result of an infection (e.g., viral infection, bacterial infection or fungal infection) causing damage to stem cells.

Immunodeficiencies, such as T and/or B lymphocytes deficiencies, or other immune disorders, such as rheumatoid arthritis and lupus, can also be treated according to the methods of the invention. Such immunodeficiencies may also be the result of an infection (for example infection with HIV leading to AIDS), or exposure to radiation, chemotherapy or toxins.

Also benefiting from treatment according to methods of the invention are individuals who are healthy, but who are at risk of being affected by any of the diseases or disorders described herein (“at-risk” individuals). At-risk individuals include, but are not limited to, individuals who have a greater likelihood than the general population of becoming cytopenic or immune deficient. Individuals at risk for becoming immune deficient include, but are not limited to, individuals at risk for HIV infection due to sexual activity with HIV-infected individuals; intravenous drug users; individuals who may have been exposed to HIV-infected blood, blood products, or other HIV-contaminated body fluids; babies who are being nursed by HIV-infected mothers; individuals who were previously treated for cancer, e.g., by chemotherapy or radiotherapy, and who are being monitored for recurrence of the cancer for which they were previously treated; and individuals who have undergone bone marrow transplantation or any other organ transplantation, or patients anticipated to undergo chemotherapy or radiation therapy or be a donor of stem cells for transplantation.

A reduced level of immune function compared to a normal subject can result from a variety of disorders, diseases infections or conditions, including immunosuppressed conditions due to leukemia, renal failure; autoimmune disorders, including, but not limited to, systemic lupus erythematosus, rheumatoid arthritis, auto-immune thyroiditis, scleroderma, inflammatory bowel disease; various cancers and tumors; viral infections, including, but not limited to, human immunodeficiency virus (HIV); bacterial infections; and parasitic infections.

A reduced level of immune function compared to a normal subject can also result from an immunodeficiency disease or disorder of genetic origin, or due to aging. Examples of these are immunodeficiency diseases associated with aging and those of genetic origin, including, but not limited to, hyperimmunoglobulin M syndrome, CD40 ligand deficiency, IL-2 receptor deficiency, γ-chain deficiency, common variable immunodeficiency, Chediak-Higashi syndrome, and Wiskott-Aldrich syndrome.

A reduced level of immune function compared to a normal subject can also, result from treatment with specific pharmacological agents, including, but not limited to chemotherapeutic agents to treat cancer; certain immunotherapeutic agents; radiation therapy; immunosuppressive agents used in conjunction with bone marrow transplantation; and immunosuppressive agents used in conjunction with organ transplantation.

In other embodiments, the methods of the invention are used to treat subjects identified as in need of an increase or decrease in angiogenesis.

Accordingly, the present invention provides methods of treating a disease and/or disorders or symptoms thereof which comprise administering a therapeutically effective amount of a pharmaceutical composition comprising a ZBP-89 polypeptide, ZBP-89 nucleic acid molecule, ZBP-89 inhibitory nucleic acid molecule, or a hematopoietic stem cell or progenitor cell thereof treated with such a polypeptide or nucleic acid molecule as described herein to a subject (e.g., a mammal, such as a human). Thus, one embodiment is a method of treating a subject having a disease characterized by a lack of blood cells or a disease wherein an alteration in the level of angiogenesis is required. Optionally, the method includes the step of administering to the mammal a therapeutic amount of a hematopoietic stem cell, progenitor cell, or mixture comprising such cell types treated with an agent that alters ZBP-89 expression or activity as described herein sufficient to treat a disease or disorder or symptom thereof, under conditions such that the disease or disorder is treated.

The methods herein include administering to the subject (including a subject identified as in need of such treatment) an effective amount of a ZBP-89 polypeptide, nucleic acid molecule, or an agent that alters ZBP-89 expression or activity to modulate the number of hematopoietic stem cells or progenitor cells or to modulate angiogenesis, or a composition described herein to produce such effect. Identifying a subject in need of such treatment can be in the judgment of a subject or a health care professional and can be subjective (e.g. opinion) or objective (e.g. measurable by a test or diagnostic method). As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated. As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

The therapeutic methods of the invention (which include prophylactic treatment) in general comprise administration of a therapeutically effective amount of a pharmaceutical composition comprising a ZBP-89 polypeptide, nucleic acid molecule, or inhibitory nucleic acid molecule, or a hematopoietic stem cell or progenitor cell, or mixture of such cell types treated with an agent that modulates ZBP-89 expression or activity, such as a compound of the formulae herein to a subject (e.g., animal, human) in need thereof, including a mammal, particularly a human. Such treatment will be suitably administered to subjects, particularly humans, suffering from, having, susceptible to, or at risk for a disease, disorder, or symptom thereof. Determination of those subjects “at risk” can, be made by any objective or subjective determination by a diagnostic test or opinion of a subject or health care provider (e.g., genetic test, enzyme or protein marker, Marker (as defined herein), family history, and the like). The compounds herein may be also used in the treatment of any other disorders in which a lack of blood cells or an alteration in angiogenesis may be implicated.

In one embodiment, the invention provides a method of monitoring treatment progress. The method includes the step of determining a level of diagnostic marker (Marker) (e.g., any target delineated herein modulated by a compound herein, a protein or indicator thereof, etc.) or diagnostic measurement (e.g., screen, assay) in a subject suffering from or susceptible to a disorder or symptoms thereof associated with having a reduced number of hematopoietic stem cells or progenitor cells, or identified as in need of an increase or decrease in angiogenesis, in which the subject has been administered a therapeutic amount of a compound herein sufficient to treat the disease or symptoms thereof. The level of Marker determined in the method can be compared to known levels of Marker in either healthy normal controls or in other afflicted patients to establish the subject's disease status. In preferred embodiments, a second level of Marker in the subject is determined at a time point later than the determination of the first level, and the two levels are compared to monitor the course of disease or the efficacy of the therapy. In certain preferred embodiments, a pre-treatment level of Marker in the subject is determined prior to beginning treatment according to this invention; this pre-treatment level of Marker can then be compared to the level of Marker in the subject after the treatment commences, to determine the efficacy of the treatment.

### Expansion of Hematopoietic Stem Cells

An increase in the number of hematopoietic stem cells (HSC) or hematopoietic stem cell progenitors can be attained by increasing expression of ZBP-89 in the cells or by contacting the cells with an agent that increases expression of a ZBP-89 polypeptide. In one preferred embodiment, the hematopoietic stem cells or their progenitors are contacted with a ZBP-89 nucleic acid molecule, such that the cells express ZBP-89. Hematopoietic stem cell progenitors include virtually any cell capable of giving rise to a hematopoietic stem cell (e.g., mesenchymal stem cells, embryonic stem cells). The hematopoietic stem cell, which may be isolated from bone marrow, blood, cord blood, fetal liver and yolk sac, is the progenitor cell that generates blood cells or following transplantation reinitiates multiple hematopoietic lineages and can reinitiate hematopoiesis for the life of a recipient. (See Fei, R., et al., U.S. Pat. No. 5,635,387; McGlave, et al., U.S. Pat. No. 5,460,964; Simmons, P., et al., U.S. Pat. No. 5,677,136; Tsukamoto, et al., U.S. Pat. No. 5,750,397; Schwartz, et al., U.S. Pat. No. 5,759,793; DiGuisto, et al., U.S. Pat. No. 5,681,599; Tsukamoto, et al., U.S. Pat. No. 5,716,827; Hill, B., et al. 1996.) When transplanted into lethally irradiated subjects (e.g., animals, humans), hematopoietic stem cells can repopulate the erythroid, neutrophil-macrophage, megakaryocyte and lymphoid hematopoietic cell pool. In vitro, hematopoietic stem cells can be induced to undergo at least some self-renewing cell divisions and can be induced to differentiate to the same lineages observed in vivo.

It is well known in the art that hematopoietic cells include pluripotent stem cells, multipotent progenitor cells (e.g., a lymphoid stem cell), and/or progenitor cells committed to specific hematopoietic lineages. The progenitor cells committed to specific hematopoietic lineages may be of T cell lineage, B cell lineage, dendritic cell lineage, Langerhans cell lineage and/or lymphoid tissue-specific macrophage cell lineage.

Where the stem cells to be provided to a subject in need of such treatment are hematopoietic stem cells, they are most commonly obtained from the bone marrow of the subject or a compatible donor. Bone marrow cells can be easily isolated using methods know in the art. For example, bone marrow stem cells can be isolated by bone marrow aspiration. U.S. Pat. No. 4,481,946, incorporated herein expressly by reference, describes a bone marrow aspiration method and apparatus, wherein efficient recovery of bone marrow from a donor can be achieved by inserting a pair of aspiration needles at the intended site of removal. Through connection with a pair of syringes, the pressure can be regulated to selectively remove bone marrow and sinusoidal blood through one of the aspiration needles, while positively forcing an intravenous solution through the other of the aspiration needles to replace the bone marrow removed from the site. The bone marrow and sinusoidal blood can be drawn into a chamber for mixing with another intravenous solution and thereafter forced into a collection bag.

U.S. Pat. No. 4,486,188 describes methods of bone marrow aspiration and an apparatus in which a series of lines are directed from a chamber section to a source of intravenous solution, an aspiration needle, a second source of intravenous solution and a suitable separating or collection source. The chamber section is capable of simultaneously applying negative pressure to the solution lines leading from the intravenous solution sources in order to prime the lines and to purge them of any air. The solution lines are then closed and a positive pressure applied to redirect the intravenous solution into the donor while negative pressure is applied to withdraw the bone marrow material into a chamber for admixture with the intravenous solution, following which a positive pressure is applied to transfer the mixture of the intravenous solution and bone marrow material into the separating or collection source.

It will be apparent to those of ordinary skill in the art that the crude or unfractionated bone marrow can be enriched for cells having desired hematopoietic stem cell characteristics. Some of the ways to enrich include, e.g., depleting the bone marrow from the more differentiated progeny. The more mature, differentiated cells can be selected against, via cell surface molecules they express. Enriched bone marrow subpopulations include but are not limited to populations sorted according to their surface expression of Lin, cKit and Sca-1 (e.g., Lin-cKit⁺Sca¹⁺). Bone marrow can be harvested during the lifetime of the subject. However, harvest prior to illness (e.g., cancer) is desirable, and harvest prior to treatment by cytotoxic means (e.g., radiation or chemotherapy) will improve yield and is therefore also desirable.

Hematopoietic stem cells can also be obtained from blood products. A “blood product” as used in the present invention defines a product obtained from the body or an organ of the body containing cells of hematopoietic origin. Such sources include unfractionated bone marrow, umbilical cord, peripheral blood, liver, thymus, lymph and spleen. It will be apparent to those of ordinary skill in the art that all of the aforementioned crude or unfractionated blood products can be enriched for cells having “hematopoietic stem cell” characteristics in a number of ways. For example, the blood product can be depleted from the more differentiated progeny. The more mature, differentiated cells can be selected against, via cell surface molecules they express. Additionally, the blood product can be fractionated selecting for CD34⁺ cells. CD34⁺ cells are thought in the art to include a subpopulation of cells capable of self-renewal and pluripotentiality. Such selection can be accomplished using, for example, commercially available magnetic anti-CD34 beads (Dynal, Lake Success, N.Y.). Unfractionated blood products can be obtained directly from a donor or retrieved from cryopreservative storage.

In one embodiment of the invention, the hematopoietic stem cells may be harvested prior to treatment with ZBP-89. “Harvesting” hematopoietic progenitor cells is defined as the dislodging or separation of cells from the matrix. This can be accomplished using a number of methods, such as enzymatic, non-enzymatic, centrifugal, electrical, or size-based methods, or preferably, by flushing the cells using media (e.g. media in which the cells are incubated). The cells can be further collected, separated, and further expanded generating even larger populations of differentiated progeny.

Stem cells of the present invention also include embryonic stem cells. The embryonic stem (ES) cell has unlimited self-renewal and pluripotent differentiation potential (Thomson, J. et al. 1995; Thomson, J. A. et al., 1998; Shamblott, M. et al. 1998; Williams, R. L. et al. 1988; Orkin, S. 1998; Reubinoff, B. E., et al. 2000). These cells are derived from the inner cell mass (ICM) of a pre-implantation blastocyst (Thomson, J. et al. 1995; Thomson, L. A. et al., 1998; Martin, G. R. 1981), or can be derived from the primordial germ cells from a post-implantation embryo (embryonal germ cells or EG cells). ES and/or EG cells have been derived from multiple species, including mouse, rat, rabbit, sheep, goat, pig and more recently from human and human and non-human primates (U.S. Pat. Nos. 5,843,780 and 6,200,806).

Embryonic stem cells are well known in the art. For example, U.S. Pat. Nos. 6,200,806 and 5,843,780 refer to primate, including human, embryonic stem cells. U.S. Patent Application Nos. 20010024825 and 20030008392 describe human embryonic stem cells. U.S. Patent Application No. 20030073234 describes a clonal human embryonic stem cell line. U.S. Pat. No. 6,090,625 and U.S. Patent Application No. 20030166272 describe an undifferentiated cell that is stated to be pluripotent. U.S. Patent Application No. 20020081724 describes what are stated to be embryonic stem cell derived cell cultures.

Stem cells of the present invention also include mesenchymal stem cells. Mesenchymal stem cells, or “MSCs” are well known in the art. MSCs, originally derived from the embryonal mesoderm and isolated from adult bone marrow, can differentiate to form muscle, bone, cartilage, fat, marrow stroma, and tendon. During embryogenesis, the mesoderm develops into limb-bud mesoderm, tissue that generates bone, cartilage, fat, skeletal muscle and endothelium. Mesoderm also differentiates to visceral mesoderm, which can give rise to cardiac muscle, smooth muscle, or blood islands consisting of endothelium and hematopoietic progenitor cells. Primitive mesodermal or MSCs, therefore, could provide a source for a number of cell and tissue types. A number of MSCs have been isolated. (See, for example, Caplan, A., et al., U.S. Pat. No. 5,486,359; Young, H., et al., U.S. Pat. No. 5,827,735; Caplan, A., et al., U.S. Pat. No. 5,811,094; Bruder, S., et al., U.S. Pat. No. 5,736,396; Caplan, A., et al., U.S. Pat. No. 5,837,539; Masinovsky, B., U.S. Pat. No. 5,837,670; Pittenger, M., U.S. Pat. No. 5,827,740; Jaiswal, N., et al., (1997). J. Cell Biochem. 64(2):295-312; Cassiede P., et al., (1996). J Bone Miner Res. 9:1264-73; Johnstone, B., et al., (1998) Exp Cell Res. 1:265-72; Yoo, et al., (1998) J Bon Joint Surg Am. 12:1745-57; Gronthos, S., et al., (1994). Blood 84:4164-73); Pittenger, et al., (1999). Science 284:143-147.

In one embodiment, the stem cell is present in a mixed population of cells, which can be purified to a degree sufficient to produce a desired effect. Those skilled in the art can readily determine the percentage of hematopoietic stem cells or their progenitors in a population using various well-known methods, such as fluorescence activated cell sorting (FACS). Purity of the stem cells can be determined according to the genetic marker profile within a population. Dosages can be readily adjusted by those skilled in the art (e.g., a decrease in purity may require an increase in dosage).

In several embodiments, it will be desirable to first purify the cells. Stem cells, including hematopoietic stem cells, of the invention preferably comprise a population of cells that have about 50-55%, 55-60%, 60-65% and 65-70% purity (e.g., non-stem and/or non-progenitor cells have been removed or are otherwise absent from the population). More preferably the purity is about 70-75%, 75-80%, 80-85%; and more preferably the purity is about 85-90%, 90-95%, and 95-100%. Purified populations of stem cells of the invention can be contacted with a ZBP-89 polypeptide or nucleic acid molecule before, after or concurrently with purification steps and administered to the subject.

### Hematopoietic Stem Cell Culture

Once obtained from a desired source, contacting of a stem cell with a ZBP-89 polypeptide or nucleic acid molecule may, if desired, occur in culture. Employing ZBP-89 polypeptide or nucleic acid molecule, it is possible to stimulate the expansion of hematopoietic stem cell number and/or colony forming unit potential. In all of the in vitro and ex vivo culturing methods according to the invention, except as otherwise provided, the media used is that which is conventional for culturing cells. Appropriate culture media can be a chemically defined serum-free media, such as the chemically defined media RPMI, DMEM, Iscove's, etc or so-called “complete media”. Typically, serum-free media are supplemented with human or animal plasma or serum. Such plasma or serum can contain small amounts of hematopoietic growth factors. The media used according to the present invention, however, can depart from that used conventionally in the prior art. Suitable chemically defined serum-free media are described in U.S. Ser. No. 08/464,599 and WO96/39487, and “complete media” are described in U.S. Pat. No. 5,486,359.

Treatment of the stem cells of the invention with a ZBP-89 polypeptide or nucleic acid molecule may involve variable parameters. For example, ex vivo treatment of stem cells with a ZBP-89 polypeptide or transfection of the cells with a ZBP-89 nucleic acid molecule may have a rapid effect or may require extended incubation periods (e.g., 24-48 hours). If desired, a hematopoietic or other stem cell may be treated according to the invention with additional agents that promote stem cell maintenance and expansion. It is well within the level of ordinary skill in the art for practitioners to vary the parameters accordingly.

The growth agents of particular interest in connection with the present invention are hematopoietic growth factors. By hematopoietic growth factors, it is meant factors that influence the survival or proliferation of hematopoietic stem cells. Growth agents that affect only survival and proliferation, but are not believed to promote differentiation, include the interleukins 3, 6 and 11, stem cell factor and FLT-3 ligand. The foregoing factors are well known to those of ordinary skill in the art and most are commercially available. They can be obtained by purification, by recombinant methodologies or can be derived or synthesized synthetically.

Thus, when cells are cultured without any of the foregoing agents, it is meant herein that the cells are cultured without the addition of such agent except as may be present in serum, ordinary nutritive media or within the blood product isolate, unfractionated or fractionated, which contains the hematopoietic stem and progenitor cells.

### Methods for Creating Genetically Altered Cells

Genetic alteration of a stem cell includes all transient and stable changes of the cellular genetic material that are created by the addition of exogenous genetic material. In one embodiment, a population of cells that includes a hematopoietic stem cell or progenitor cell is transfected with a ZBP-89 nucleic acid molecule to enhance ZBP-89 expression and increase the number of hematopoietic stem cells or progenitor cells in a culture or a subject. In an alternate embodiment, a ZBP-89 nucleic acid molecule or a ZBP-89 inhibitory nucleic acid molecule (e.g., siRNA, shRNA, antisense oligonucleotides) is used to modulate (e.g., increase, reduce) angiogenesis in a tissue in need of such modulation. In one approach, an inhibitory nucleic acid molecule is introduced directly into a target cell, tissue, or organ, such that the inhibitory nucleic acid molecule reduces expression of ZBP-89 in the cell, tissue, or organ. In another approach, the target cell is transduced with an expression vector that encodes a ZBP-89 inhibitory nucleic acid molecule, such that expression of the inhibitory nucleic acid molecule reduces expression of ZBP-89 in the cell, tissue, or organ. Expression of the ZBP-89 inhibitory nucleic acid molecule in the target cell reduces ZBP-89 expression. A reduction in the expression in ZBP-89 is expected to increase angiogenesis in the target cell, tissue, or organ. Increased angiogenesis is useful for the treatment of conditions where an increase in blood flow to the target tissue or organ is desirable. Such conditions include, but are not limited to, tissues affected by an ischemic injury, such as cardiac ischemia, myocardial infarction, stroke, and intermittent diabetic claudication.

Alternatively, the target cell, tissue or organ is transduced with an expression vector that encodes a ZBP-89 polypeptide. Expression of the ZBP-89 encoding nucleic acid molecule increases ZBP-89 polypeptide expression in the cell, tissue, or organ. Overexpressing ZBP-89 is expected to increase expression of scl (B), lmo2 (D), and gata-1 (F), and to reduce expression of the endothelial markers flk1 (II) and tie1 (J). Desirably, ZBP-89 overexpression is expected to increase the number of CD45⁺ hematopoietic progenitors cells. Ectopic expression of ZBP-89 is expected to reduce angiogenesis. Accordingly, an increase in expression of ZBP-89 is useful for the treatment of conditions characterized by an undesirable increase in angiogenesis. Such conditions include, but are not limited to, neoplasia (e.g., glioblastoma and renal cell carcinoma) and ocular conditions that exhibit an increase in choroidal neovascularization (e.g., age-related macular degeneration, diabetic retinopathy, ischemic retinopathy).

Various techniques may be employed for introducing nucleic acids into cells. Such techniques include transfection of nucleic acid-CaPO₄ precipitates, transfection of nucleic acids associated with DEAE, transfection with a retrovirus including the nucleic acid of interest, liposome mediated transfection, and the like. For certain uses, it is preferred to target the nucleic acid to particular cells. In such instances, a vehicle used for delivering a nucleic acid according to the invention into a cell (e.g., a retrovirus, or other virus; a liposome) can have a targeting molecule attached thereto. For example, a molecule such as an antibody specific for a surface membrane protein on the target cell or a ligand for a receptor on the target cell can be bound to or incorporated within the nucleic acid delivery vehicle. For example, where liposomes are employed to deliver the nucleic acids of the invention, proteins which bind to a surface membrane protein associated with endocytosis may be incorporated into the liposome formulation for targeting and/or to facilitate uptake. Such proteins include proteins or fragments thereof tropic for a particular cell type, antibodies for proteins which undergo internalization in cycling, proteins that target intracellular localization and enhance intracellular half life, and the like. Polymeric delivery systems also have been used successfully to deliver nucleic acids into cells, as is known by those skilled in the art. Such systems even permit oral delivery of nucleic acids.

One method of introducing exogenous genetic material into cells involves transducing the cells in situ on the matrix using replication-deficient retroviruses. Replication-deficient retroviruses are capable of directing synthesis of all virion proteins, but are incapable of making infectious particles. Accordingly, these genetically, altered retroviral vectors have general utility for high-efficiency transduction of genes in cultured cells, and specific utility for use in the method of the present invention. Retroviruses have been used extensively for transferring genetic material into cells. Standard protocols for producing replication-deficient retroviruses (including the steps of incorporation of exogenous genetic material into a plasmid, transfection of a packaging cell line with plasmid, production of recombinant retroviruses by the packaging cell line, collection of viral particles from tissue culture media, and infection of the target cells with the viral particles) are provided in the art.

Because viruses insert efficiently a single copy of the gene encoding the therapeutic agent into the host cell genome, retroviruses permit the exogenous genetic material to be passed on to the progeny of the cell when it divides. In addition, gene promoter sequences in the LTR region have been reported to enhance expression of an inserted coding sequence in a variety of cell types. However, using a retrovirus expression vector may result in (1) insertional mutagenesis, i.e., the insertion of the therapeutic gene into an undesirable position in the target cell genome which, for example, leads to unregulated cell growth and (2) the need for target cell proliferation in order for the therapeutic gene carried by the vector to be integrated into the target genome. Despite these apparent limitations, delivery of a therapeutically effective amount of a therapeutic agent via a retrovirus can be efficacious if the efficiency of transduction is high and/or the number of target cells available for transduction is high.

Yet another viral candidate useful as an expression vector for transformation of cells is the adenovirus, a double-stranded DNA virus. Like the retrovirus, the adenovirus genome is adaptable for use as an expression vector for gene transduction, i.e., by removing the genetic information that controls production of the virus itself. Because the adencivirus functions usually in an extrachromosomal fashion, the recombinant adenovirus does not have the theoretical problem of insertional mutagenesis. On the other hand, adenoviral transformation of a target cell may not result in stable transduction. However, more recently it has been reported that certain adenoviral sequences confer intrachromosomal integration specificity to carrier sequences, and thus result in a stable transduction of the exogenous genetic material.

Thus, as will be apparent to one of ordinary skill in the art, a variety of suitable vectors are available for transferring exogenous genetic material into cells. The selection of an appropriate vector to deliver an agent and the optimization of the conditions for insertion of the selected expression vector into the cell, are within the scope of one of ordinary skill in the art without the need for undue experimentation. The promoter characteristically has a specific nucleotide sequence necessary to initiate transcription. Optionally, the exogenous genetic material further includes additional sequences (i.e., enhancers) required to obtain the desired gene transcription activity. For the purpose of this discussion an “enhancer” is simply any nontranslated DNA sequence which works contiguous with the coding sequence (in cis) to change the basal transcription level dictated by the promoter. Preferably, the exogenous genetic material is introduced into the cell genome immediately downstream from the promoter so that the promoter and coding sequence are operatively linked so as to permit transcription of the coding sequence. A preferred retroviral expression vector includes an exogenous promoter element to control transcription of the inserted exogenous gene. Such exogenous promoters include both constitutive and inducible promoters.

Naturally-occurring constitutive promoters control the expression of important cell functions. As a result, a gene under the control of a constitutive promoter is expressed under all conditions of cell growth. Exemplary constitutive promoters include the promoters for the following genes which encode certain constitutive or “housekeeping” functions: hypoxanthine phosphoribosyl transferase (HPRT), dihydrofolate reductase (DHFR) (Scharfmann et al., 1991, Proc. Natl. Acad. Sci. USA, 88:4626-4630), adenosine deaminase, phosphoglycerol kinase (PGK), pyruvate kinase, phosphoglycerol mutase, the actin promoter (Lai et al., 1989, Proc. Natl. Acad. Sci. USA, 86:10006-10010), and other constitutive promoters known to those of skill in the art. In addition, many viral promoters function constitutively in eukaryotic cells. These include: the early and late promoters of SV40; the long terminal repeats (LTRS) of Moloney Leukemia Virus and other retroviruses; and the thymidine kinase promoter of Herpes Simplex Virus, among many others. Accordingly, any of the above-referenced constitutive promoters can be used to control transcription of a heterologous gene insert.

Genes that are under the control of inducible promoters are expressed only or to a greater degree, in the presence of an inducing agent, (e.g., transcription under control of the metallothionein promoter is greatly increased in presence of certain metal ions). Inducible promoters include responsive elements (REs) which stimulate transcription when their inducing factors are bound. For example, there are REs for serum factors, steroid hormones, retinoic acid and cyclic AMP. Promoters containing a particular RE can be chosen in order to obtain an inducible response and in some cases, the RE itself may be attached to a different promoter, thereby conferring inducibility to the recombinant gene. Thus, by selecting the appropriate promoter (constitutive versus inducible; strong versus weak), it is possible to control both the existence and level of expression of an agent in the genetically modified cell. Selection and optimization of these factors for delivery of an is deemed to be within the scope of one of ordinary skill in the art without undue experimentation, taking into account the above-disclosed factors.

In addition to at least one promoter and at least one heterologous nucleic acid, the expression vector preferably includes a selection gene, for example, a neomycin resistance gene, for facilitating selection of cells that have been transfected or transduced with the expression vector. Alternatively, the cells are transfected with two or more expression vectors, at least one vector containing the gene(s) encoding the therapeutic agent(s), the other vector containing a selection gene. The selection of a suitable promoter, enhancer, selection gene and/or signal sequence is deemed to be within the scope of one of ordinary skill in the art without undue experimentation. In preferred embodiments, expression of a ZBP-89 nucleic acid molecule is under the control of a human flk1 promoter or other endothelial cell specific promoters, β-actin promoter in embryonic stem cells, or smooth muscle actin for cardiac myocytes.

### Methods of Using Inhibitory Nucleic Acids

The inhibitory nucleic acid molecules of the present invention may be employed as double-stranded RNAs for RNA interference (RNAi)-mediated knock-down of ZBP-89 expression. In one approach, ZBP-89 expression is reduced in a stem cell (e.g., a hemangioblast or embryonic stem cell) to alter the cell fate. In one exemplary approach, ZBP-89 expression is reduced in a target cell, tissue or organ. RNAi is a method for decreasing the cellular expression of specific proteins of interest (reviewed in Tuschl, Chembiochem 2:239-245, 2001; Sharp, Genes & Devel. 15:485-490, 2000; Hutvagner and Zamore, Curr. Opin. Genet. Devel. 12:225-232, 2002; and Hannon, Nature 418:244-251, 2002). The introduction of siRNAs into cells either by transfection of dsRNAs or through expression of siRNAs using a plasmid-based expression system is increasingly being used to create loss-of-function phenotypes in mammalian cells.

In one embodiment of the invention, double-stranded RNA (dsRNA) molecule is made that includes between eight and twenty-five consecutive nucleobases of a 0.10 nucleobase oligomer of the invention. The dsRNA can be two distinct strands of RNA that have duplexed, or a single RNA strand that has self-duplexed (small hairpin (sh)RNA). Typically, dsRNAs are about 21 or 22 base pairs, but may be shorter or longer (up to about 29 nucleobases) if desired. dsRNA can be made using standard techniques (e.g., chemical synthesis or in vitro transcription). Kits are available, for example, from Ambion (Austin, Tex.) and Epicentre (Madison, Wis.). Methods for expressing dsRNA in mammalian cells are described in Brummelkamp et al. Science 296:550-553, 2002; Paddison et al. Genes & Devel. 16:948-958, 2002. Paul et al. Nature Biotechnol. 20:505-508, 2002; Sui et al. Proc. Natl. Acad. Sci. USA 99:5515-5520, 2002; Yu et al. Proc. Natl. Acad. Sci. USA 99:6047-6052, 2002; Miyagishi et al. Nature Biotechnol. 20:497-500, 2002; and Lee et al. Nature Biotechnol. 20:500-505 2002, each of which is hereby incorporated by reference.

Small hairpin RNAs consist of a stem-loop structure with optional 3′ UU-overhangs. While there may be variation, stems can range from 21 to 31 by (desirably 25 to 29 bp), and the loops can range from 4 to 30 by (desirably 4 to 23 bp). For expression of shRNAs within cells, plasmid vectors containing either the polymerase III H1-RNA or U6 promoter, a cloning site for the stem-looped RNA insert, and a 4-5-thymidine transcription termination signal can be employed. The Polymerase III promoters generally have well-defined initiation and stop sites and their transcripts lack poly(A) tails. The termination signal for these promoters is defined by the polythymidine tract, and the transcript is typically cleaved after the second uridine. Cleavage at this position generates a 3′ UU overhang in the expressed shRNA, which is similar to the 3′ overhangs of synthetic siRNAs. Additional methods for expressing the shRNA in mammalian cells are described in the references cited above.

### Delivery of Nucleobase Oligomers

Naked inhibitory nucleic acid molecules, or analogs thereof, are capable of entering mammalian cells and inhibiting expression of a gene of interest. Nonetheless, it may be desirable to utilize a formulation that aids in the delivery of oligonucleotides or other nucleobase oligomers to cells (see, e.g., U.S. Pat. Nos. 5,656,611, 5,753,613, 5,785,992, 6,120,798, 6,221,959, 6,346,613, and 6,353,055, each of which is hereby incorporated by reference).

### Administration of Cells of the Invention

Hematopoietic stem cells, progenitor cells, or a mixture comprising such cell types are administered according to methods known in the art. Such compositions may be administered by any conventional route, including injection or by gradual infusion over time. The administration may, depending on the composition being administered, for example, be, pulmonary, intravenous, intraperitoneal, intramuscular, intracavity, subcutaneous, or transdermal. The stem cells are administered in “effective amounts”, or the amounts that either alone or together with further doses produces the desired therapeutic response.

Administered cells of the invention can be autologous (“self”) or non-autologous (“non-self,” e.g., allogeneic, syngeneic or xenogeneic). Generally, administration of the cells can occur within a short period of time following ZBP-89 polypeptide or nucleic acid molecule treatment (e.g. 1, 2, 5, 10, 24 or 48 hours after treatment) and according to the requirements of each desired treatment regimen. For example, where radiation or chemotherapy is conducted prior to administration, treatment, and transplantation of stem cells of the invention should optimally be provided within about one month of the cessation of therapy. However, transplantation at later points after treatment has ceased can be done with derivable clinical outcomes.

### Stem Cell Related Pharmaceutical Compositions

Following harvest and treatment with a suitable ZBP-89 polypeptide or nucleic acid molecule, hematopoietic stem cells or their progenitors, or a mixture of cells that include these cells may be combined with pharmaceutical excipients known in the art to enhance preservation and maintenance of the cells prior to administration. In some embodiments, cell compositions of the invention can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propyleneglycol, liquid polyethylene glycol, and the like) and suitable mixtures thereof.

Sterile injectable solutions can be prepared by incorporating the cells utilized in practicing the present invention in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like.

The compositions can be isotonic, i.e., they can have the same osmotic pressure as blood and lacrimal fluid. The desired isotonicity of the compositions of this invention may be accomplished using sodium chloride, or other pharmaceutically acceptable agents such as dextrose, boric acid, sodium tartrate, propylene glycol or other inorganic or organic solutes. Sodium chloride is preferred particularly for buffers containing sodium ions.

A method to potentially increase cell survival when introducing the cells into a subject in need thereof is to incorporate stem cells of interest into a biopolymer or synthetic polymer. Depending on the subject's condition, the site of injection might prove inhospitable for cell seeding and growth because of scarring or other impediments. Examples of biopolymer include, but are not limited to, cells mixed with fibronectin, fibrin, fibrinogen, thrombin, collagen, and proteoglycans. This could be constructed with or without included expansion or differentiation factors. Additionally, these could be in suspension, but residence time at sites subjected to flow would be nominal. Another alternative is a three-dimensional gel with cells entrapped within the interstices of the cell biopolymer admixture. Again, expansion or differentiation factors could be included with the cells. These could be deployed by injection via various routes described herein.

Those skilled in the art will recognize that the components of the compositions should be selected to be chemically inert and will not affect the viability or efficacy of the stem cells or their progenitors as described in the present invention. This will present no problem to those skilled in chemical and pharmaceutical principles, or problems can be readily avoided by reference to standard texts or by simple experiments (not involving undue experimentation), from this disclosure and the documents cited herein.

One consideration concerning the therapeutic use of stem cells is the quantity of cells necessary to achieve an optimal effect. Different scenarios may require optimization of the amount of cells injected into a tissue of interest. Thus, the quantity of cells to be administered will vary for the subject being treated. The precise determination of what would be considered an effective dose may be based on factors individual to each patient, including their size, age, sex, weight, and condition of the particular patient. As few as 100-1000 cells can be administered for certain desired applications among selected patients. Therefore, dosages can be readily ascertained by those skilled in the art from this disclosure and the knowledge in the art.

The skilled artisan can readily determine the amount of cells and optional additives, vehicles, and/or carrier in compositions and to be administered in methods of the invention. Of course, for any composition to be administered to an animal or human, and for any particular method of administration, it is preferred to determine therefore: toxicity, such as by determining the lethal dose (LD) and LD₅₀ in a suitable animal model e.g., rodent such as mouse; and, the dosage of the composition(s), concentration of components therein and timing of administering the composition(s), which elicit a suitable response. Such determinations do not require undue experimentation from the knowledge of the skilled artisan, this disclosure and the documents cited herein. And, the time for sequential administrations can be ascertained without undue experimentation.

### Pharmaceutical Compositions Related to Cell Fate Alteration

The invention provides a simple means for identifying compositions (including nucleic acids, peptides, small molecule inhibitors, and mimetics) capable of acting as therapeutics by increasing the number of hematopoietic stem cells or modulating angiogenesis. In particular, the invention provides ZBP-89 polypeptide or nucleic acid molecule compositions or analogs, or mimetics thereof that are useful for expansion of a hematopoietic stem cell population or modulation of angiogenesis. Accordingly, a chemical entity discovered to have medicinal value using the methods described herein is useful as a drug or as information for structural modification of existing compounds, e.g., by rational drug design. For therapeutic uses, the compositions or agents identified using the methods disclosed herein may be administered systemically, for example, formulated in a pharmaceutically-acceptable buffer such as physiological saline. Preferable routes of administration include, for example, subcutaneous, intravenous, interperitoneally, intramuscular, or intradermal injections that provide continuous, sustained levels of the drug in the patient. Treatment of human patients or other animals will be carried out using a therapeutically effective amount of a therapeutic in a physiologically-acceptable carrier. Suitable carriers and their formulation are described, for example, in Remington's Pharmaceutical Sciences by E. W. Martin. The amount of the therapeutic agent to be administered varies depending upon the manner of administration, the age and body weight of the patient, and with the clinical symptoms of the disease. Generally, amounts will be in the range of those used for other agents used in the treatment of other diseases associated with expansion of a hematopoietic stem cell population or modulation of angiogenesis. A compound is administered at a dosage that controls the clinical or physiological symptoms of a disease as determined by a diagnostic method known to one skilled in the art, or using any that assay that measures the expression or the biological activity of an ZBP-89 polypeptide or nucleic acid molecule.

### Screening Assays

Screening methods of the invention can involve the identification of compound that promotes the expansion of a population of hematopoietic stem cells or that modulates angiogenesis. Such methods will typically involve contacting a population of cells with a candidate compound in culture and quantitating the number of hematopoietic stem cells produced as a result. Alternatively, the cells contacted may include cells capable of expressing an endothelial cell marker or undergoing angiogenesis (e.g., vessel formation, sprouting) and marker expression or angiogenesis following contact with the candidate marker is quantitated. Comparison to an untreated control can be concurrently assessed. Where an increase in the number of hematopoietic cells or an alteration in the expression of an endothelial cell marker or an alteration in angiogenesis is detected relative to the control, the candidate compound is determined to have the desired activity.

Increased amounts of hematopoietic stem cells can be detected by an increase in gene expression of certain markers including, but not limited to, Scl, Lmo2, Gata-1, CD34, rnx-1, Hes-1, Bmi-1, Gfi-1, SLAM genes, CD51, GATA-2, GATA-3, P2y14. These cells may also be characterized by a decreased or low expression of genes associated with endothelial cell differentiation (e.g., endothelial markers Flk1 and Tie1). Alternatively, where an increase in angiogenesis is desired, a compound of the invention increases the expression of an endothelial cell marker or increases angiogenesis. The level of expression of genes of interest can be measured in a number of ways, including, but not limited to: measuring the mRNA encoded by the genes; measuring the amount of protein encoded by the genes; or measuring the activity of the protein encoded by the genes.

The level of mRNA corresponding to a gene of interest can be determined both by in situ and by in vitro formats. The isolated mRNA can be used in hybridization or amplification assays that include, but are not limited to, Southern or Northern analyses, polymerase chain reaction analyses and probe arrays. One diagnostic method for the detection of mRNA levels involves contacting the isolated mRNA with a nucleic acid molecule (probe) that can hybridize to the mRNA encoded by the gene being detected. The nucleic acid probe is sufficient to specifically hybridize under stringent conditions to mRNA or genomic DNA. The probe can be disposed on an address of an array, e.g., an array described below. Other suitable probes for use in the diagnostic assays are described herein.

In one format, mRNA (or cDNA) is immobilized on a surface and contacted with the probes, for example by running the isolated mRNA on an agarose gel and transferring the mRNA from the gel to a membrane, such as nitrocellulose. In an alternative format, the probes are immobilized on a surface and the mRNA (or cDNA) is contacted with the probes, for example, in a two-dimensional gene chip array described below. A skilled artisan can adapt known mRNA detection methods for use in detecting the level of mRNA encoded by the genes of interest described herein.

The level of mRNA in a sample can be evaluated with nucleic acid amplification, e.g., by reverse transcription-polymerase chain reaction (rtPCR) (Mullis (1987) U.S. Pat. No. 4,683,202), ligase chain reaction (Barmy (1991) Proc. Natl. Acad. Sci. USA 88:189-193), self sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197), rolling circle replication (Lizardi et al., U.S. Pat. No. 5,854,033) or any other nucleic acid amplification method, followed by the detection of the amplified molecules using techniques known in the art. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

For in situ methods, a cell or tissue sample can be prepared/processed and immobilized on a support, typically a glass slide, and then contacted with a probe that can hybridize to mRNA that encodes the gene of interest being analyzed.

In another aspect, screening methods of the invention may be used to identify compositions that induce hematopoietic stem cell expansion by enhancing the expression or activity of a ZBP-89 polypeptide or nucleic acid molecule. Alternatively, screening methods of the invention may be used to identify compositions that reduce angiogenesis by altering the expression or activity of a ZBP-89 polypeptide or nucleic acid molecule. In one embodiment, the candidate compound is a compound that reduces expression of a ZBP-89 polypeptide in a hemangioblast.

Compositions and methods of the present invention are directed towards the treatment of expanding a hematopoietic stem cell population or for modulating angiogenesis in a subject. Such treatment is sought by administering a XBP-89 nucleic acid molecule, polypeptide, or inhibitory nucleic acid molecule, or a functional mimetic thereof to a subject, by effecting an increase or decrease in the expression of XBP-89 in a subject, or by inhibiting XBP-89 expression or activity in a subject. In certain embodiments, XBP-89 is over-produced in hematopoietic cells, endothelial cells, or their progenitors of the subject. Thus, the administration of XBP-89, increase in XBP-89 expression, or inhibition of XBP-89 expression or activity seeks increase the number of hematopoietic stem cells or modulate angiogenesis in the subject.

In certain embodiments, XBP-89 or a functional mimetic there of has at least about 60%, 70% 80% 90% 95%, 99%, or 100% sequence identity with an exemplary human XBP-89 polypeptide, or a fragment thereof. In additional embodiments of the present invention, methods are contemplated that identify agonists or antagonists of XBP-89 expression or activity. Design of assays (e.g., cell-based assays) to identify such agonists and antagonists is well within the skill in the art. The end point of the assays will typically measure protein expression or a physiologic effect.

### Test Compounds and Extracts

In general, compounds capable of modulating the expression or activity of a ZBP-89 polypeptide are identified from large libraries of both natural product or synthetic (or semi-synthetic) extracts or chemical libraries or from polypeptide or nucleic acid libraries, according to methods known in the art. Those skilled in the field of drug discovery and development will understand that the precise source of test extracts or compounds is not critical to the screening procedure(s) of the invention. Compounds used in screens may include known compounds (for example, known therapeutics used for other diseases or disorders). Alternatively, virtually any number of unknown chemical extracts or compounds can be screened using the methods described herein. Examples of such extracts or compounds include, but are not limited to, plant-, fungal-, prokaryotic- or animal-based extracts, fermentation broths, and synthetic compounds, as well as modification of existing compounds.

Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, chemical compounds to be used as candidate compounds can be synthesized from readily available starting materials using standard synthetic techniques and methodologies known to those of ordinary skill in the art. Synthetic chemistry transformations and protecting group methodologies (protection and deprotection) useful in synthesizing the compounds identified by the methods described herein are known in the art and include, for example, those such as described in R. Larock, Comprehensive Organic Transformations, VCH Publishers (1989); T. W. Greene and P. G. M. Wuts, Protective Groups in Organic Synthesis, 2nd ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods. Examples of methods for the synthesis of molecular libraries can be found in the art, for example in: DeWitt et al., Proc. Natl. Acad. Sci. U.S.A. 90:6909, 1993; Erb et al., Proc. Natl. Acad. Sci. USA 91:11422, 1994; Zuckermann et al., J. Med. Chem. 37:2678, 1994; Cho et al., Science 261:1303, 1993; Carrell et al., Angew. Chem. Int. Ed. Engl. 33:2059, 1994; Carell et al., Angew. Chem. Int. Ed. Engl. 33:2061, 1994; and Gallop et al., J. Med. Chem. 37:1233, 1994. Furthermore, if desired, any library or compound is readily modified using standard chemical, physical, or biochemical methods.

Libraries of compounds may be presented in solution (e.g., Houghten, Biotechniques 13:412-421, 1992), or on beads (Lam, Nature 354:82-84, 1991), chips (Fodor, Nature 364:555-556, 1993), bacteria (Ladner, U.S. Pat. No. 5,223,409), spores (Ladner U.S. Pat. No. 5,223,409), plasmids (Cull et al., Proc Natl Acad Sci USA 89:1865-1869, 1992) or on phage (Scott and Smith, Science 249:386-390, 1990; Devlin, Science 249:404-406, 1990; Cwirla et al. Proc. Natl. Acad. Sci. 87:6378-6382, 1990; Felici, J. Mol. Biol. 222:301-310, 1991; Ladner supra.).

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their activity should be employed whenever possible.

When a crude extract is found to modulate the expression or activity of a ZBP-89 polypeptide, further fractionation of the positive lead extract is necessary to isolate chemical constituents responsible for the observed effect. Thus, the goal of the extraction, fractionation, and purification process is the careful characterization and identification of a chemical entity within the crude extract that decreases the expression or activity of an ZBP-89 polypeptide. Methods of fractionation and purification of such heterogenous extracts are known in the art. If desired, compounds shown to be useful as therapeutics for supporting stem cell expansion are chemically modified according to methods known in the art.

### Kits

The invention provides kits for promoting hematopoietic stem cell expansion, as well as kits for modulating angiogenesis. In one embodiment, the kit includes a therapeutic composition containing an effective amount of ZBP-89 polypeptide or nucleic acid molecule or ZBP-89 inhibitory nucleic acid molecule in unit dosage form. In one example, an effective amount of ZBP-89 is an amount sufficient to promote hematopoietic stem cell expansion in vitro or in vivo. Alternatively, an effective amount of ZBP-89 is an amount sufficient to modulate (e.g., increase or decrease) angiogenesis in a target cell, tissue, or organ.

In other embodiments, the kit comprises a sterile container which contains a therapeutic or prophylactic vaccine; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

If desired a ZBP-89 polypeptide or nucleic acid molecule or an agent that modulates the activity thereof is provided together with instructions for administering it to a culture or to a subject. The instructions will generally include information about the use of the composition for the expansion of a stem cell population in culture or in the subject. In other embodiments, the instructions include at least one of the following: description of the ZBP-89 polypeptide or nucleic acid molecule; dosage schedule and administration for the expansion of a stem cell population or the modulation of angiogenesis; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

### Administration of Cells

Hematopoietic stem cells, progenitor cells, or a mixture comprising such cell types are administered to a subject according to methods known in the art. Such compositions may be administered by any conventional route, including injection or by gradual infusion over time. The administration may; depending on the composition being administered, for example, be, pulmonary, intravenous, intraperitoneal, intramuscular, intracavity, subcutaneous, or transdermal. The stem cells are administered in “effective amounts”, or the amounts that either alone or together with further doses produces the desired therapeutic response.

Administered cells of the invention can be autologous (“self”) or non-autologous (“non-self,” e.g., allogeneic, syngeneic or xenogeneic). Generally, administration of the cells can occur within a short period of time following ZBP-89 polypeptide or nucleic acid molecule treatment (e.g. 1, 2, 5, 10, 24 or 48 hours after treatment) and according to the requirements of each desired treatment regimen. For example, where radiation or chemotherapy is conducted prior to administration, treatment, and transplantation of stem cells of the invention should optimally be provided within about one month of the cessation of therapy. However, transplantation at later points after treatment has ceased can be done with derivable clinical outcomes.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1

### Knockdown of ZBP-89 Produces a Bloodless Phenotype in Zebrafish Embryos

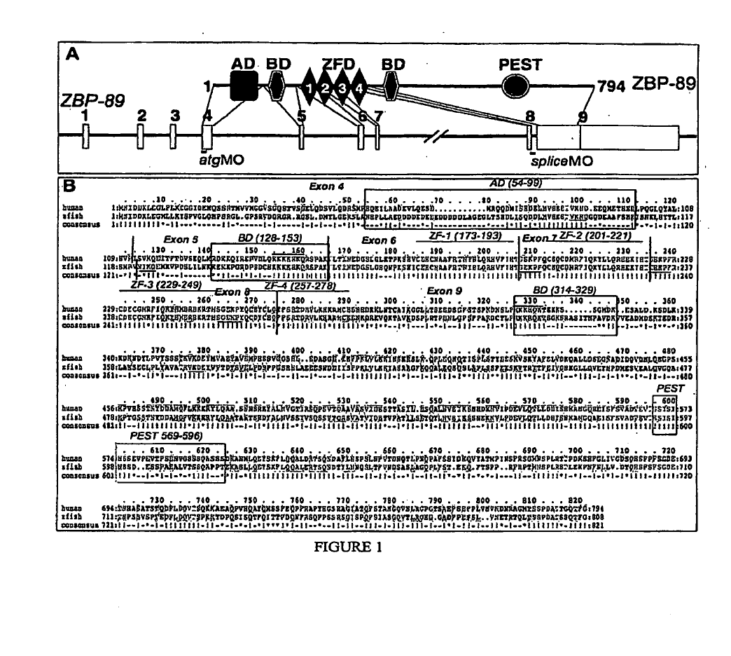

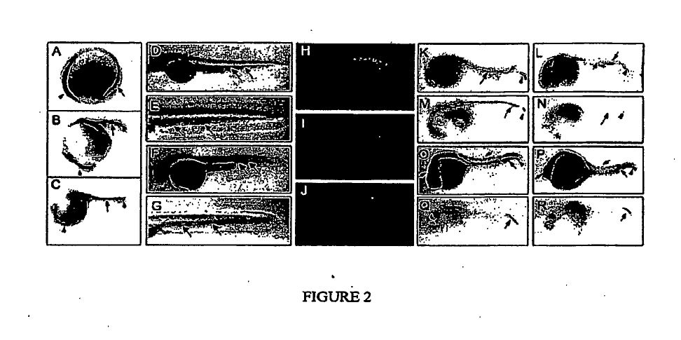

The ex utero development of zebrafish embryos allows the accurate determination of the early embryonic origins of the adult hematopoietic system before the onset of circulation. To explore its role in hematopoiesis in vivo, the zebrafish ortholog of ZBP-89 (FIGS. 1A and 1B) was cloned. Zebrafish ZBP-89 shares a 74.1% similarity and 61.8% amino acid identity with human ZBP-89. Whole-mount in situ hybridization showed that ZBP-89 is expressed in the posterior lateral mesoderm, the head mesenchyme, and in the intermediate cell mass (ICM, equivalent to the yolk sac in mammals) of 24 hour postfertilization (hpf) zebrafish embryos (FIG. 2A), an expression pattern resembling that of scl (Liao et al., 1998).

Antisense morpholinos (MO) targeting the translation start site (atgMO) or splice donor site (spliceMO) in exon 8 in ZBP-89, the latter causing a splicing defect, were used to knockdown expression of this gene in developing zebrafish embryos. MO oligos are named for the morpholino-based neutral-charge backbone replacing the phosphate or phosphorothioate backbones in regular antisense oligos and act as sequence-specific translational inhibitors or as steric blockers of RNA splicing, depending on the targeted sequence. Translational blockade by MO oligo injection in zebrafish is effective for at least the first 50 hours of development, well within the time period of organogenesis.

No blood cells were observed either within or outside the vasculature in either atgMO- or spliceMO-injected live 48hpf embryos (FIG. 2B), although all embryos displayed a beating heart. Over time, pericardial edema developed in MO-injected embryos, presumably due to the absence of blood circulation, and the morphants began to exhibit axis deformities, with the vast majority dying four days post fertilization (dpf). Zebrafish 1-2 cell embryos injected with MO containing five mismatches in atgMO (mismatched atgMO) developed normally (FIG. 2B). Subsequent analyses were carried out in the atgMO-generated morphants. The bloodless phenotype caused by depletion of ZBP-89 was rescued by co-injecting atgMO with WT human ZBP-89 mRNA into WT embryos or with a plasmid in which ZBP-89 is expressed under control of the flk1 promoter into Tg (gata1:GFP) embryos (FIG. 2C).

### Example 2

### ZBP-89-Depleted Zebrafish Embryos Fail to Develop Primitive or Definitive Blood Hematopoiesis

In zebrafish, primitive hematopoiesis arises from two regions of the lateral mesoderm: the anterior lateral mesoderm located rostrally in the head region that gives rise to the myeloid lineage, and the posterior lateral mesoderm, which forms the intermediate cell mass (ICM, equivalent of the extraembryonic mammalian yolk sac blood island) just ventral to the notochord, where erythroid development takes place (Al-Adhami and Kunz, 1977; Fouquet et al., 1997; Gering et al., 1998; Herbomel et al., 1999). Expression of the early hematopoiesis markers scl, lmo2 and gata-2 (Liao et al., 1998) was reduced in 12-24 hpf embryos depleted of ZBP-89 (FIGS. 2D, B, D and F, respectively). This loss was not caused by defects in mesodermal conversion into blood and blood vessel precursors, as reflected by the normal expression of the caudal hox-related gene cdx4 (Davidson et al., 2003) (FIGS. 2D, G-H). Whole-mount TUNEL staining of ZBP-89 morphants did not reveal a significant increase in apoptosis of cells in the embryonic blood island and tail bud region in 22hpf embryos compared to WT, indicating that ZBP-89 may be required for fate specification rather than survival of early hematopoietic precursors (FIGS. 2A-2R).

### Example 3

### Effect of ZBP-89 Knockdown on Early Hematopoietic Markers in Zebrafish

Classic loss-of-function/gain-of-function approaches were used to assess the impact of modifying ZBP-89 levels on hematopoietic and vascular stem cell development in zebrafish. To investigate the role of ZBP-89 in primitive hematopoiesis, the effect of ZBP-89 knockdown on expression of the early hematopoietic genes scl, lmo2 and gata2 was examined. Expression of scl in the anterior and posterior lateral plate mesoderm marks the specification of primitive hematopoietic progenitors at 11hpf (Liao et al., 1998). By 24 hpf, scl mRNA is found in the anterior and posterior ICM (FIG. 2A-R). Scl also contributes to formation of the axial vein of the posterior tail and to definitive hematopoiesis. Lmo2 is required in blood progenitor differentiation (Warren et al., 1994). Its tissue distribution closely follows that of scl (FIG. 2A-R), with which it interacts. Gata2 is involved in hematopoietic progenitor proliferation (Fujiwara et al., 2004). It is expressed in ventral mesoderm and hematopoietic progenitor cells (Detrich et al., 1995; Minegishi et al., 1999), and by 24 hours, its expression was confined to the hematopoietic and neuronal tissues including the primordial lateral line ganglia and spinal secondary motor neurons (FIG. 2A-R)(Nardelli et al., 1999).

### Example 4

### Effect of ZBP-89 Loss on Primitive Erythroid and Myeloid Markers in Zebrafish

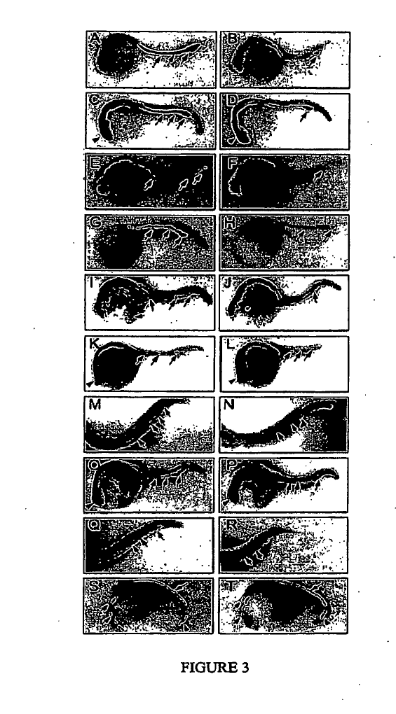

ZBP-89-depleted embryos failed to develop primitive and definitive hematopoiesis. Expression of the primitive erythroid markers gata-1 and tif1g (moonshine) (Ransom et al., 2004) in the ICM was lost almost entirely in ZBP-89 morphants (FIG. 3, B, D, respectively). However, expression of tif1g in the central nervous system was unaffected (FIG. 3, C, D), reflecting the specificity of the atgMO-induced defects to the regions of active hematopoiesis/HSC formation. Expression of the primitive myeloid lineage markers pu.1, mpo and l-plastin was also markedly downregulated in 24hpf ZBP-89 morphants (FIGS. 3, F, H and J, respectively). Thus, ZBP-89 depletion reproduced the primitive hematopoietic (both erythroid and myeloid) defects seen in scl null mice (Porcher et al., 1996; Robb et al., 1996), as well as in zebrafish scl morphants (Patterson et al., 2005) (FIGS. 3A-3T).

### Example 5

### Loss of ZBP-89 Blocks Definitive Hematopoiesis

Definitive hematopoiesis in zebrafish embryos occurs by 32 hpf in the ventral wall of the dorsal aorta, a region equivalent to the mammalian aorta-gonad-mesonephros (AGM) region (Burns et al., 2002; Kalev-Zylinska et al., 2002). The expression of the definitive hematopoiesis markers runx1 and c-myb, ablation of either one of which results in complete absence of definitive hematopoiesis (Burns et al., 2002; Mucenski et al., 1991; Okuda et al., 1996), was examined. Expression of runx1 and c-myb was reduced by depletion of ZBP-89 in 24hpf and 48 hpf zebrafish embryos (FIG. 3, K-R). This was particularly evident in the stem cell population associated with the dorsal aorta; the few cells that continued to express these markers were mostly confined to the posterior-most portion of the ICM. Loss of ZBP-89 did not affect expression of runx1 or c-myb in non-hematopoietic tissues (FIG. 3L, P). Thus, ZBP-89 depletion phenocopies the defects in primitive and definitive hematopoiesis seen in scl null mice (Porcher et al., 1996; Robb et al., 1996), as well as in scl zebrafish morphants (Patterson et al., 2005). Expression of fli1a and flk1, indicative of primary blood vessel formation, was minimally affected by loss of ZBP-89 in 12-20hpf embryos (FIG. 3, S-T).

Expression of both markers was dramatically increased in 48hpf embryos. Thus, the hematopoietic defects were similar to the loss-of-function scl phenotype, but the marked increase in angiogenesis seen in zbp-89 morphants at 48hpf is different from that seen with scl loss-of-function (Dooley et al., 2005), reflecting opposing roles of ZBP-89 on hematopoietic and angioblast precursors.

### Example 6

### ZBP-89 Acts Downstream of Clo but Upstream of Scl

The clo gene product is required in primitive and definitive hematopoiesis, as well as for development of the vascular system (clo^{-/-} embryos lack fli1 and flk1 in the anterior ICM). To position ZBP-89 in the regulatory gene cascade leading to blood formation, human ZBP-89 mRNA was injected into 1-2-cell stage embryos collected from the clo^{fv087b} mapping cross.

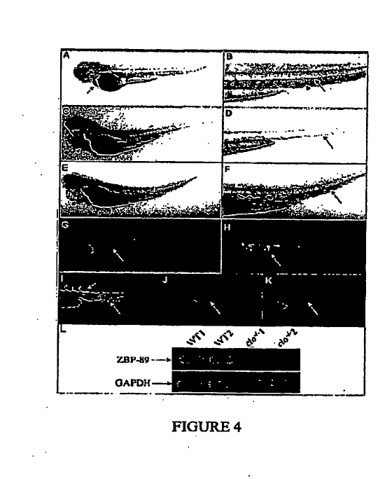

The injected embryos were fixed at 3 dpf and stained with DAF. Embryos with positive DAF-staining were genotyped with the SSR marker z1496, very tightly linked to clo^{fv087b} (FIGS. 4A-4K). Rescue of the hematopoietic program, detected by DAF staining, was observed in homozygous clo mutant embryos (FIG. 4E, f), indicating that ZBP-89 overexpression can rescue hematopoiesis in the complete absence of clo function. This finding was also confirmed by overexpressing ZBP-89 in embryos from fv087b^{+/-} clo Tg (gata1:GFP) crosses. As shown in FIG. 4H, this treatment rescued the GATA1⁺ cell population in the anterior ICM.

To assess if forced expression of ZBP-89 also reconstitutes blood vessel formation, a transgenic zebrafish line, Tg (flk1:EGFP), in which the flk1 promoter is directing expression of EGFP (Cross et al., 2003), was used. Flk1:EGFP was expressed in the blood vasculature of WT embryos (FIG. 4I), but was not expressed in clo embryos from fv087b^{+/-}.:Tg (flk1:EGFP) crosses (FIG. 4J). Injection of ZBE-89 mRNA into 1-cell stage embryos from such crosses did not rescue flk1 expression (FIG. 4K). Thus, ZBP-89 also acted functionally downstream of clo but in contrast to SCL was able to rescue the hematopoietic, but not the vascular lineage. Next, the ZBP-89 transcript levels were evaluated in clo mutants by RT-PCR. As shown in FIG. 4L, ZBP-89 mRNA was significantly reduced in clo null mutant embryos compared to wild-type embryos, consistent with the above functional data.

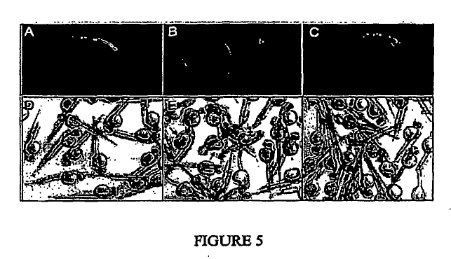

To evaluate the functional relationship of ZBP-89 with SCL, scl RNA was co-injected with atgMO into 1-2 stage transgenic Tg (gata1:EGFP) zebrafish embryos, where EGFP is under control of the gata-1 promoter, a line that strictly labels erythrocytes (Long et al., 1997). scl RNA was found to rescue GATA1 expression in 22hpf embryos, as well as pericardial edema and circulating blood in 48hpf embryos (FIGS. 5A-5F) and the axial deformities in 3 dpf embryos. Thus, ZBP-89 acts upstream of scl in the transcriptional hierarchy of early hematopoiesis.

### Example 7

### Ectopic Expression of ZBP-89 in WT Zebrafish Embryos Expands the Hematopoietic Markers but Impairs Vascular Remodeling

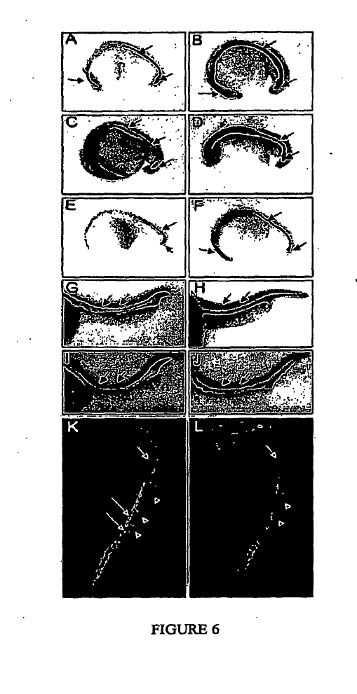

Forced expression of ZBP-89 in wild-type zebrafish embryos caused significant expansion of the early hematopoietic markers scl-, lmo2- and gata-1 (FIGS. 6B, D and F, respectively). Expression levels of the WT1 and pax2.1 markers of the pronephros and pronephric duct, respectively (Gering et al., 2003), were unchanged, indicating that ectopic ZBP-89 expression did not change the fates of kidney mesoderm in the early lateral mesoderm. The strong induction of early hematopoietic markers indicates that ZBP-89 has a dominant role in commitment to the hematopoietic lineage (FIGS. 6A-6L). Ectopic (over)expression of ZBP-89 induced a simultaneous reduction in flk1 (FIGS. 6H, L) and Tie1 (FIG. 6J) expression in the intersomitic and axial blood vessels in 24hpf embryos compared to control (FIG. 6G, I, K).

### Example 8

### Expression Profile of ZBP-89 in ESCs, Hemangioblasts; Hematopoietic and Angioblast Progenitors

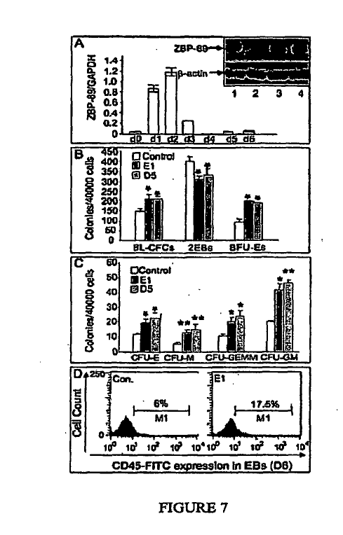

To assess if ZBP-89 is also important for hematopoietic development in mammals, expression of ZBP-89 was analyzed in mouse embryonic stern cells (ESCs) undergoing differentiation into hematopoietic stem cells, and the consequences of its stable overexpression on hematopoietic and vascular development in vitro was determined. The ZBP-89 transcript was not detected in undifferentiated ESCs, but it was rapidly induced in early (day-1) EBs, peaking in day-2 EBs then declining afterwards (FIG. 7A).

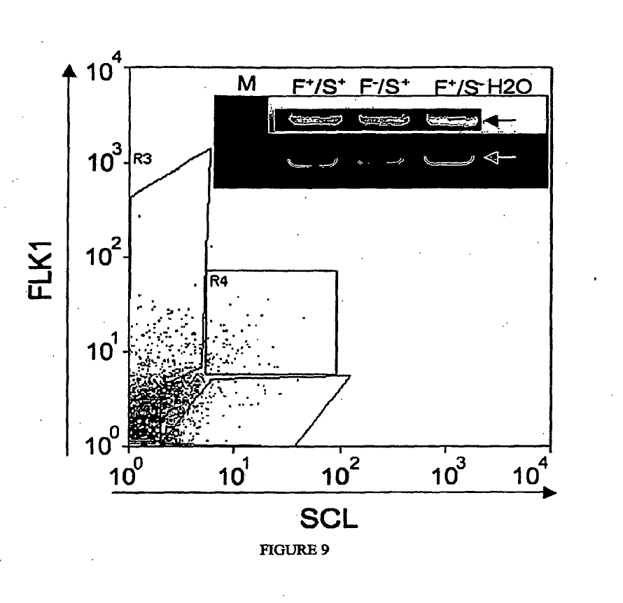

Under similar conditions, expression of the early hematopoietic markers runx1, scl and gata1 begins at or after day-3 of culture (Lacaud et al., 2002), indicating that in the mouse, as in zebrafish, ZBP-89 also acts upstream of these factors. The ZBP-89 protein was prominently expressed in day-3 EB-derived FLK1⁺ mesoderm precursors (FIG. 7A, inset), and its transcript was also present in the FLK1⁺SCL⁺ hemangioblasts (BL-CFC), FLK1⁻SCL⁺ hematopoietic progenitors and in FLK1⁺ angioblasts derived from day-4 EBs (FIG. 9).

### Example 9

### Overexpression of ZBP-89 in Mouse ESCs Leads to Increased Hematopoiesis but Reduced Sprouting Angiogenesis

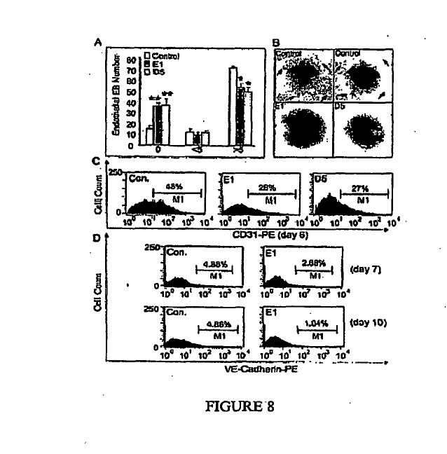

The role of ZBP-89 in hematopoietic and endothelial lineage commitment was evaluated using the mouse ES cell in vitro differentiation system. Stable ectopic expression of ZBP-89 in ESC/EBs lead to a significant increase in the hemangioblast (blast colony forming cell, BL-CFC) population (FIG. 7B). Stable overexpression of ZBP-89 in ESC/EBs also induced a 5-fold increase in the number of SCA-1⁺/c-KIT⁺ hematopoietic progenitors in 3-day- and 4-day EB cultures, and a 2-fold expansion of primitive erythroid colonies (BFU-Es)(FIG. 7B), definitive erythrocyte (CFU-E), macrophage (CFU-M), granulocyte-macrophage-megakaryocyte (CFU-GEMM), and granulocyte-macrophage (CFU-GM) colonies (Kennedy et al., 1997)(FIG. 7C), as well as a 3-fold increase in the number of the cell population expressing the hematopoietic marker CD45 (FIG. 7D). In contrast to the expansion of HSC precursors by forced expression of ZBP-89, formation of the vascular plexus (Feraud et al., 2001) by the cultured ZBP-89-overexpressing EBs was significantly reduced (FIG. 8A, B), as was the number of the CD31⁺ (FIG. 8C) and VE-Cadherin⁺ (FIG. 8D) endothelial cell population.

The kinetics of expression of ZBP-89 mRNA during normal ESC differentiation was studied in murine EB liquid cultures at days 1-6 using real time PCR (Li et al., 2006). ZBP-89 mRNA was not expressed in undifferentiated ES J1 cells (day-0, D0), but was rapidly induced in early EBs (day-1, D1), peaking in day-2 (D2) then declining afterwards. To evaluate ZBP-89 protein expression by immunoblotting, EB-derived cell lysates were electroblotted then probed with goat anti-mouse ZBP-89 polyclonal antibody (Santa Cruz), anti-goat IgG-horseradish peroxidase (HRP)-conjugated secondary antibody, and visualized using the ECL kit (BioRad). A mouse monoclonal anti-β-actin antibody (Sigma) was used to document equal loading. BL-CFCs were generated from EBs in ESC differentiation medium (StemCell Technologies). The ZBP-89 protein was not detected in undifferentiated ESCs but was found abundantly in day 3 FLK1⁺ progenitors and to a much lesser degree in the FLK1⁻ population.

The expression of ZBP-89 in the early HSC and angioblast populations was examined, making use of the hCD4-scl murine ES cells that were generated by knock-in of the non-functional human CD4 receptor into the SCL locus (Chung et al., 2002), thus allowing quantitative counting of SCL⁺ cells by FACS analysis with the anti-hCD4 monoclonal antibody. hCD4-SCL ESCs were differentiated into EBs in a suspension culture for 4-days, immunostained for FLK1 and CD4, and sorted into FLK1⁺SCL⁺ (clo⁺) hemangioblasts, FLK1⁻SCL⁺ (clo⁻) HSCs, FLK1⁺SCL⁻ (clo⁺) angioblasts. These cells were present at 1.5-5%, 13-23% and 4-10% of the total population, respectively. Semi quantitative (sq) RT-PCR analysis (30 cycles) of RNA from each population is shown in FIG. 9.

Mouse ZBP-89 expression in sorted cells from the FLK⁺SCL⁺, FLK⁻SCL⁺, and FLK⁺SCL⁻ population was carried out as follows: 1×10⁵ cells from each subset were collected, total RNA extracted and cDNA synthesis done as described above. RT-PCR was performed using the mouse ZBP-89 forward primer: 5′-GAGATTTCCTTCAGCGTTTAC-3′ and reverse primer: 5′-TTTGGAAGGGTCTGGTTGTC-3′.

ZBP-9 was found to be expressed in hemangioblasts, as well as in HSCs and angioblasts, indicating that the differential effects of ZBP-89 on growth or differentiation of the respective cell population may be driven by the nature of DNA binding complexes formed by ZBP-89 in each the respective cell lineages.

### Example 10

### Knockdown of ZBP-89 in ESCs Markedly Reduces FLK1⁺ Precursors in EB Cultures

Short hairpin RNAs were used to knockdown the wild-type ZBP-89 allele in the ZBP-89^{lacZ/+} ES cell line. 21-mers encoding 5 different shRNAs (112-6), for specific siRNA-based post-transcriptional silencing of mouse ZBP-89, were each cloned into the pRRL plasmid, which contains a puromycin marker, and the plasmid incorporated into lentivirus using the helper and packaging system pΔD8.9, pMD.G (VSV-G) (Invitrogen). Virus particles in CM DMEM medium were then used to infect ZBP-89^{lacZ/+} ESC line by placing 3×10⁴ cells in 10% FBS/DMEM in individual wells of a tissue culture treated 96-well plate. Polybrene (8 μg/ml) and 20 μl of virus containing medium (˜1.5×10³/μl) at a low multiplicity of infection (MOI (of 1 were added to each well and the cells incubated at 37° C. overnight in a 100 μl total volume in humidified 5% CO2 incubator. Plates were centrifuged at 1,500 rpm for 5 minutes and the supernatant was removed. Fresh medium (100 μL of 10% FBS/IMDM) was added to each well and the plate incubated at 37° C.

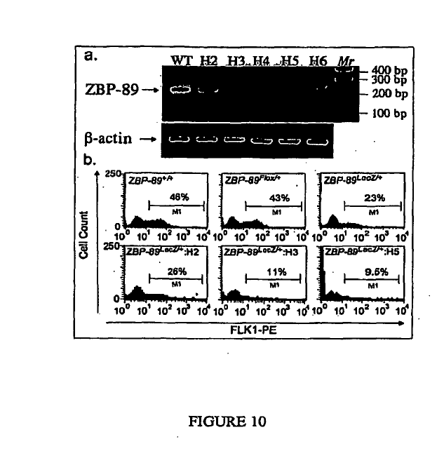

After 36-48 h, lentivirus-infected cells were selected from non-infected cells by adding puromycin (2.5 μg/ml) in the growth medium for one week, with a change of media at 3-day intervals. Puromycin-resistant clones were isolated and tested for down regulation of WT ZBP-89 allele by semiquantitative (sq)-RT-PCR and Western blotting. Two clones (H3 and H5 that showed ˜75% and 95% overall silencing of the WT ZBP-89 allele, and clone H2 that showed minimal suppression by sqRT-PCR, FIG. 10A), and WT ESCs were used to generate EBs for further analysis. Successful siRNA-mediated knockdown of the wild-type allele in ZBP-89^{lacZ/+} ESCs in clones H3 and H5 (but not in clone H2) led to a dramatic drop in FLK1⁺ progenitors from day 4 EB cultures (FIG. 10B).

### Example 11

### ZBP-89 Knockdown in ESCs Reduced Murine Hematopoiesis In Vitro

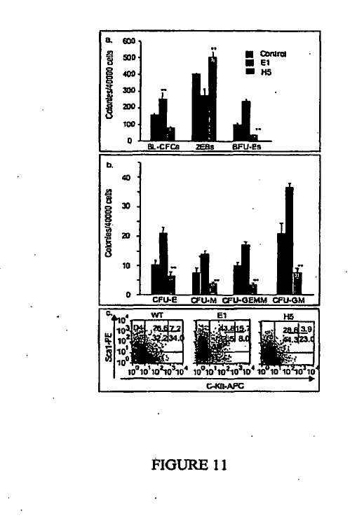

The significant drop in the number of FLK1⁺ progenitors derived from ZBP-89^{lacZ/-} ESCs prompted investigation of the effect of ZBP-89 deficiency on in vitro early hematopoiesis. Day 4 BL-CFC hemangioblast cell population was significantly reduced in EBs derived from ESC clone H5, when compared to WT control or E1 clone overexpressing ZBP-89 (FIG. 11A). There was a corresponding increase in the number of secondary EBs in the ZBP-89-deficient H5 clone (FIG. 11A). The numbers of primitive erythroid and myeloid colonies (FIG. 11B), as well as the number of the SCA-1+/c-KIT+ hematopoietic progenitors (FIG. 11C), were also significantly reduced compared to the WT or to ZBP-89 overexpressing EBs. There was a corresponding major reduction in the number of CD45⁺ hematopoietic cells in ESC H5 progeny.

### Example 12

### Stable ZBP-89 Knockdown in ESCs Markedly Increased Sprouting Angiogenesis in Murine EB Cultures In Vitro

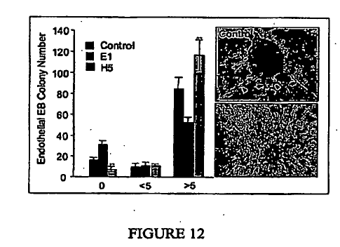

Knockdown of ZBP-89 exerted dramatic enhancing effects on angiogenesis in murine EB cultures (FIG. 12). These results are opposite to the inhibitory effects observed when ZBP-89 was ectopically expressed in EBs (FIG. 8). The changes were also mirrored in the significant increase in angioblast markers CD31 and VE-cadherin, both of which are suppressed in ESCs ectopically expressing ZBP-89 (FIG. 8).

### Example 13

### Generation of ESCs and Mice Heterozygous for a Floxed ZBP-89 Allele

In view of the early embryonic lethality of ZBP-85^{-/-} mice and the lack of fertility of ZBP-89^{+/-} male mice, generation of mice in which ZBP-89 is conditionally knocked out is useful for investigating its role in adult hematopoiesis. ESCs and mice heterozygous for a ZBP-89 loxP-flanked (foxed, fox) allele were generated using the Cre-loxP sequence-specific recombination system (Gu et al., 1994). The ZBP-89^{flox/+} genotype was verified in ESCs and mice, and the proper excision of the recombinant locus was demonstrated.

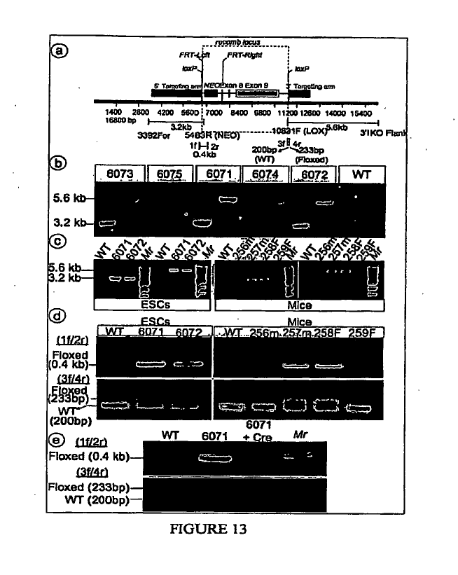

Cre-induced recombination should result in deletion of exons 8 and 9 (that encode ZF3, ZF4 and the bulk of the protein). This deletion was engineered to resemble the previous embryonically-lethal knockout resulting from deletion of exon 9 (which encodes half of ZF4 and the long C-terminus of the protein that contains a strong transcriptional activation domain). The added deletion of ZF3-4 in the conditional knockout minimizes DNA binding of the residual protein if expressed, and deletion of exon 9 results in ≧50% reduction in mRNA levels encoding the mutant allele) (Takeuchi et al., 2003). The conditional loss of ZBP-89 function in the gastrointestinal tract is examined by mating with vimentin promoter-driven Cre mice. The generation of the ESCs and mice is described in FIGS. 13A-13E.

An inducible knockout (IKO) targeting vector, PLM10, was constructed targeting exons 8 and 9 for eventual removal, and carrying the positive selection Neo resistance gene (flanked by FRT sites allowing for cleavage and removal of the cassette in the presence of FLP recombinase, in case its presence interferes with ZBP-89 expression), and the TK and DT-A cassettes (diphtheria toxin A chain) for negative selection. The plasmid was electroporated into Bruce4 ES cells, a C57BL/6 stem cell line. Because the resulting mice are on a pure C57BL/6 background, they do not require backcrossing. Clones that demonstrated presence of the homologous arms with selection cassette and loxP sequence were selected for expansion. Homologous recombination was confirmed using PCR and oligonucleotides both external and internal to the 5′ and 3′ recombination sites (FIG. 13). When cultured in parallel with wild-type ESCs (ZBP-89^{+/+}), and ZBP-89^{LacZ/+}ESCs (heterozygous for ZBP-89 null allele), ESC clone #6071 produced the same number of FLK1⁺ cells at day 4 EB cultures (FIG. 13), indicating that the recombination event and the presence of Neo resistance gene did not affect normal expression of the floxed ZBP-89 allele.

When the Tet-ON-regulated Cre recombinase was transiently expressed in ZBP-89^{flox/+} ESCs using the lentivirus system, ZBP-89⁻⁺ ESCs were generated, as judged by PCR analysis of genomic DNA before and after addition of tetracycline (FIG. 13E). Prior to injection, the ES cells were tested for euploidy, and the targeting plasmid was re-confirmed. ES cells were injected into blastocysts harvested from a C57BL/6 albino female and then reimplanted into another albino animal. Chimeras resulting from the implantation demonstrate black and white coat color due to the genetic contribution from the albino blastocysts cells and the black stem cells. Chimeras were bred back to C57BL/6 albino animals, and germline transmission was demonstrated by solid black coat color in the resulting pups. The pups were then tested by PCR for the presence of the NEO cassette to show heterozygosity for the targeted allele (FIG. 13C). One male (257m) and one female (258F) ZBP-89^{flox/+} mouse (FIG. 13C) are used for breeding.

### Example 14

### Characterization of Mice Homozygous for a Hypomorphic Mutant ZBP-89 Allele

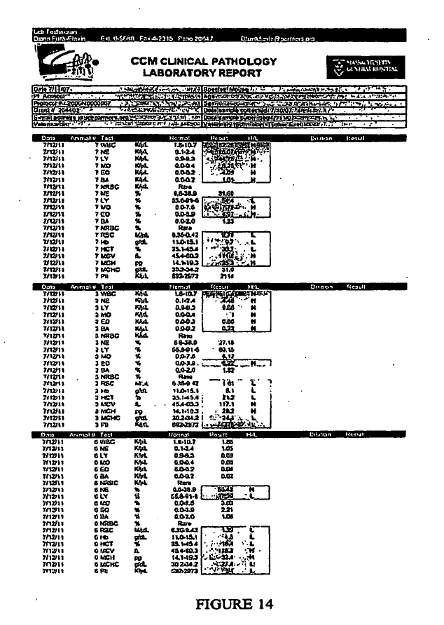

The ZBP-89^{flox/flox} mice have been observed to develop normally, but die perinatally. Survival of these mice into the perinatal period, indicates that the ZBP-89^{flox} allele is hypomorphic. Since this allele retained the Neo gene, it is likely that aberrant splicing involving the Neo results in an inactive form of ZBP-89 together with low quantities of the correctly spliced form, thus ensuring survival of some newborn mice. Crosses of the phenotypically-normal ZBP-89^{flox/+} mice with flp-Cre mice were carried out to remove the Neo gene altogether. The peripheral blood count was evaluated from several homozygous mice. The results showed a dramatic reduction in the erythroid series (FIG. 14).

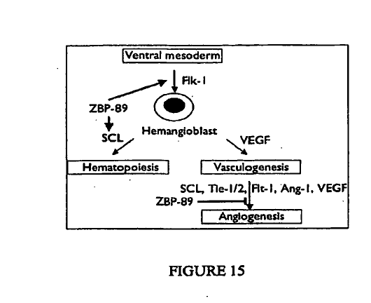

The data described above indicate a central role of ZBP-89 in hemangioblast fate determination (FIG. 15). It acts at least at two nodes: one in inducing expression of SCL, which triggers HSC differentiation and the other at the level of angioblasts, suppressing differentiation along this pathway.

Unless otherwise indicated, the results described above were obtained using the following methods and materials.

### Fish Strain and Maintenance

Breeding zebrafish were raised, maintained, and staged as described (Westerfield, 1993). The cloche^{m39} zebrafish mutant lines, gata1:EGFP and fLk1:EGFP transgenic fishes were described elsewhere (Long et al., 1997; Stainier et al., 1995).

### Cloning of the Zebrafish ZBP-89 Full Length Coding Sequence

Zebrafish ZBP-89 was cloned by RT-PCR based on the sequence predicted from Sanger Center's zebrafish genomic DNA sequence (Sanger Institute), using the human ZBP-89 protein sequence as bait. The zebrafish ZBP-89 full-length coding sequence was all contained in Contig #25012. The predicted exon/intron boundary was obtained using the website genes.mit.edu/genscan.html. A series of PCR primers was designed accordingly, and nested RT-PCR reactions were performed using total RNA from 24hpf zebrafish embryos.

A 1.3 kb cDNA encoding the N-terminal fragment of ZBP-89 was generated in first-round RT-PCR with the primers F1: 5′-TGCTGGAGGACATGAATCCACCAG-3′ and R1: 5′-TGGAGAGAGACTCTGGGACTGCTC-3′. The Qiagen-gel purified fragment was used as template DNA in a nested PCR reaction using enzyme-restricted (underlined) primers EcoRI-F: 5′-AAGAGAATTCATGAACATTGATGACAAGCTGG-3′ and XbaI-R: 5′-CTGCTCTAGAGCCTGCTG-3′. The nested PCR products were cloned into the EcoRI-XbaI-restricted PSK⁺ vector (PSK-1.3-ZBP-89) and were sequenced. The 1.1 kb encoding the C-terminal fragment of ZBP-89 was cloned following a similar strategy: the first round primers were F2: 5′-TCCCCACCTGGCAGCAGGCATCTTG-3′ and R2: 5-AGCTTTTGTTCA GCCAAAGGTTTG-3′, and the nested PCR primers were XbaI-F: 5′-CAGCAGGCTCTAGAG CAG-3″ and NotI-R: 5-AAGAGCGGCCGCTCAGCCAAAGGTTTGGCT-3′. The 1.1 kb C-terminal fragment was inserted into the PSK-1.3-ZBP-89 vector to reconstitute the cDNA encoding the full-length protein.

Zebrafish ZBP-89 shares a 74.1% similarity and 61.8% amino acid identity with human ZBP-89.

Morpholino Oligonucleotide, mRNA and Plasmid Microinjection

Three morpholino antisense oligonucleotides (MO) targeting the ZBP-89 transcript were obtained from Gene-Tools, LLC. One oligomer (atgMO, 5′-CCTCCAGCTTGTCATCAATGTTCAT-3′) was designed to block translation of the in RNA, leading to knockdown of the protein. A second (spliceMO, 5′-GTCAAAATATTACCTGATGGCAATA-3′) targeted an exon splice donor site in exon 8. A third MO contained a five-nucleotide mismatch in atgMO (Mismatched atgMO, 5′-CCaCCtGCTTGTgATCAATcTTgAT-3′, mismatched bases are in small letters). Each morpholino oligomer was diluted in 100 mmol/l KCl, 10 mmol/l HEPES, 0.1% Phenol Red (Sigma). Zebrafish embryos were microinjected at the 1-2 cell stage with 4 ng in 2 n1 of the morpholino oligomer and allowed to develop to 48-60 hpf. One control for specificity was embryos injected with 4 ng of mismatch atgMO. A second control were embryos injected with spliceMO. Rescue experiments were done by co-injection of capped RNA (cRNA) together with a MO. cRNA was made using mMESSAGE mMACHINE (Ambion). For the injection of the cRNA or cRNA together with MO, a microprocessor-controlled nanoliter injector (Nanoliter 2000, World Precision Instruments) was used.

RT-PCR analysis revealed formation of an alternative splice product in spliceMO-injected embryos that was predominant at 24hpf and encoded a 237 amino acid ZBP-89 protein terminating with Thr231 of exon 7C (after the second zing finger domain) followed by five out-of-frame residues.

The full-length human ZBP-89 cDNA was directionally cloned into EcoRI and Xho I of pCS2⁺ for overexpression. Human ZBP-89 sense RNA and zebrafish scl sense RNA (Gering et al., 1998) were transcribed from linearized pCS2⁺-ZBP-89 using mMessenge mMachine according to manufacturer's protocol (Ambion). Clo^{-/-} mutant embryos for microinjection were obtained from heterozygous in-crosses, and 100 pg of ZBP-89 mRNA was injected in 1-2 cell embryos. The full-length human ZBP-89 coding region was, subcloned downstream of zebrafish flk1 promoter (Cross et al., 2003) using EcoRI and NotI sites to generate the flk1-ZBP-89 plasmid. For morpholino rescue experiments, 300 pg of RNA or 4 ng of the linearized flk1-ZBP89 plasmid DNA were injected in 1-2 cell embryos immediately following the morpholino injection.

### Whole-Mount In Situ Hybridization

A 2.4 kb EcoR I-Not I-restricted zebrafish ZBP-89 fragment was cloned into the PSK⁺ vector. Antisense mRNA was transcribed from the EcoRI-Linearized plasmid using T3 polymerase, and sense mRNA were transcribed from the NotI-linearized plasmid using T7 polymerase as a control. RNA antisense probes were generated with UTP-digoxygenin (DIG) according to the manufacturer's instructions (Roche). Antisense riboprobes to gata-1, scl, lmo2, c-myb, flk1, fi1a, Tie1, Cdx4, tif1g (moonshine), gata-2, runx1, pu.1, l-plastin and mpo have been described previously (Ransom et al., 2004) (Davidson et al., 2003) (Kalev-Zylinska et al., 2002; Liao et al., 1998; Thompson et al., 1998){Lyons, 1998 #5726}. DIG-labeled riboprobes were detected using alkaline phosphatase conjugated anti-DIG antibodies (Roche), followed by detection of alkaline phosphatase activity using NBT/BCIP substrate (Roche).

### TUNEL Assay

Zebrafish embryos were staged and fixed as for in situ hybridization and stored in methanol. After rehydration, embryos were permeabilized by proteinase K digestion, re-fixed in buffered 4% PFA, washed in PBT. Apoptosis was detected in embryos by terminal transferase dUTP nick-end labeling (TUNEL), according to the manufacturer's protocols (In Situ Cell Death Detection Kit: POD, Roche). The staining pattern was observed using light microscopy.

### DAF Staining

36hpf embryos were stained with DAF (2,7-diaminofluorene), which sensitively stains haemoglobin, as described (Weinstein et al., 1996). In brief, embryos were fixed in 4% PFA for 2 hours, washed 3 time in PBS, then preincubated in the DAF staining solution (0.01% diaminofluorene, 200 mM Tris, pH 7.0, 0.05% Tween 20) for 1 hour at room temperature in the dark. 30% hydrogen peroxide was added to a final concentration of 0.3%, and the embryos were incubated for 5-20 minutes. Embryos were washed 3 times in PBS then photographed.

### RT-PCR Analysis of ZBP-89 Expression

A single wild-type (WT) and a clo^{-/-} mutant zebrafish embryo were harvested at the 18hpf stage, rinsed twice with PBS and transferred into an RNase-Free tube containing 100 μl RNA. Total RNA was extracted according to the manufacturer's instructions (Ambion). 4 μl of total RNA were used as a template for cDNA synthesis following the Applied Biosystems manual. 2 μl from a total of 50 μl cDNA were used for regular RT-PCR with the zebrafish ZBP-89 forward primer: 5′-GAAAAGCCTTTCCAGTGCAATCA-3′ and reverse primer: 5′-ATCTTTGACAGCTGTITCTGCAC-3′.

### ESC Culture, Differentiation and Colony Assays

J1 ES cells were maintained as described (Wang et al., 2004). HCD4-scl mouse ES cells (kindly provided by Dr. K. Choi at Wash U) (Chung et al., 2002) that were generated by knock-in of the non-functional human CD4 receptor into the SCL locus thus allowing quantitative counting of SCL⁺ cells by FACS analysis with the anti-hCD4 monoclonal antibody. Mouse ESC clones D5 and E1 that stably overexpress ZBP-89, and a control stable ES cell line were generated by transfecting the linearized plasmid encoding mouse ZBP-89 under control of the β-actin promoter (for the D5 and E1 ESCs) or the vector alone (control ESC), followed by selection in neomycin. The resulting ES cell lines were maintained on the mouse feeder cell line SNL in ES medium containing Dulbecco's modified Eagle medium (DMEM), 10 ng/ml mouse leukemia inhibitory factor (mLIF; Chemicon International, Temecula, Calif.), 15% fetal calf serum (FCS; HyClone, Logan, Utah), 1 mM sodium pyruvate, 2 mM glutamine, 0.1 mM nonessential acid, 100 μM monothioglycerol (MTG, Sigma, St Louis, Mo.), 50 U/ml penicillin, and 50 μg/ml streptomycin. ESCs were cultured to about 50% confluence on gelatin-coated plates prior to EB induction.

ESC differentiation into EB and colony assays was carried out as described (Wang et al., 2004). Briefly, EBs were generated either in liquid or 1% methylcellulose cultures (1×10⁴ ES cells per 35-mm Petri dish) in ESC differentiation medium containing Iscove Modified Dulbecco's medium (IMDM), 15% FCS, 2 mM Glutamine, 450 μM MTG, 50 μg/ml ascorbic acid and 20% BIT (1% bovine serum albumin [BSA], 10 μg/ml insulin, and 200 μg/mL transferring (StemCell Technologies). BL-CFCs were counted from EBs at 4 days of culture. To generate blast colonies from hemangioblasts, 1×10⁴/ml EBs were replated on 35 mm petri dishes in 1% methylcellulose in the presence of IMDM, 2 mM glutamine 450 μM MTG, 25 μg/ml ascorbic acid, 20% BIT, 5 ng/ml human vascular endothelial growth factor (hVEGF), 50 ng/ml SCF, 10 ng/ml human fibroblast growth factor (hFGF-2), and 2 U/ml hEPO. BL-CFCs can be recognized as loose clusters of cells after 4 days of culture. Primitive erythroid progenitors were obtained from day-6 EBs. Definitive myeloid progenitors were obtained from day 10-12 EBs in 1% methylcellulose matrix. Hematopoietic colonies were counted 7-10 days after replating. For vascular-like EB, culture, EBs were initially generated in 1% methylcellulose cultures for 11 days, transferred into collagen matrix for 3 days then examined.

### Real-Time PCR

One million FLK1⁺ mesoderm stem cells were dissociated from EBs at different time points rinsed twice with PBS and transferred into an RNase-Free tube containing 100 μl RNA. Total RNA isolation and cDNA synthesis were carried out as described in the previous section. 2 μl from a total of 50 μl cDNA were used for real time PCR that was performed according to the manufacturer's instructions (Stratagene). The ZBP-89 real time PCR primers were: forward primer, ZBP-89RF1: 5′-CGGCATAGACGAAATGCAGTC-3′; the reverse primer ZBP-89RR1,5′-CCTGGTGAGGCAAACTTCGAT-3′. The internal control primers were: GAPDHF: 5′-TGACCACAGTCCATGCCATC-3′. GAPDHR: 5′-GACGGACACATTGGGGGTTAG-3′.

### Western Blotting

Embryoid bodies were lysed with RIPA buffer (50 mM Tris-HCl, pH 7.4, 150 mM NaCl [TBS], 1% NP-40, 0.5% sodium deoxycholate, 0.1% SDS, freshly protease inhibitor cocktail (Roche)) for 30 minutes on ice then spun for 10 minutes at 10,000 rpm. Samples were loaded onto 7.5% SDS-polyacrylamide gels together with molecular weight markers (Invitrogen), and transferred to nitrocellulose membrane. The blots were incubated with goat polyclonal IgG antibody to the conserved C-terminus of human ZBP-89 (Santa Cruz), diluted 1:200 in TBS containing 0.05% Tween-20 overnight and washed extensively. After incubating with a donkey anti-goat IgG-horseradish peroxidase (HRP)-conjugated secondary antibody (Dako) (diluted 1:2000) at room temperature for 1 hour, the blots were visualized using the ECL kit (BioRad) according to the manufacturer's instructions. A mouse monoclonal anti-β-actin antibody (Sigma) at a dilution of 1:500 was used to document equal loading per lane.

### Immunostaining, Flow Cytometric Analysis and Sorting

Single cell suspensions were prepared from embryoid bodies cultured for different time periods by trypsinization of embryoid body cultures for 2 minutes at 37° C., then passed through a 21-gauge needle. Cells were immunostained (15 minutes, 4° C. in PBS/0.1% BSA buffer) with phycoerythrin (PE)- or allophycocyanin (APC)-rat anti-mouse monoclonal antibodies against c-KIT (APC), FLK1 (PE), SCA1 (PE), CD45 (FITC), embryoid bodies, CD31 (PE) (PharMingen, Becton, San Diego, Calif.), or, in the case of CD4-scl ESCs, with an additional biotinylated mouse monoclonal antibody to human CD4 (CALTAG) followed by streptavidin-APC (Sav-APC; Pharmingen). Cells stained with anti-VE-cadherin were visualized using a secondary PE-labeled goat anti-rat IgG. Cells were then analyzed using FACS Caliber or sorted using FACS MoFlo (Becton Dickinson).

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

### REFERENCES

Al-Adhami, M. A. and Kunz, Y. W. (1977). Ontogenesis of haematopoietic sites in brachydanio rerio. Dev. Growth. Differ. 19, 171-179.
Antona, V., Cammarata, G., De Gregorio, L., Dragani, T. A., Giallongo, A. and Feo, S. (1998). The gene encoding the transcriptional repressor BERF-1 maps to a region of conserved synteny on mouse chromosome 16 and human chromosome 3 and a related pseudogene maps to mouse chromosome 8. Cytogenet Cell Genet. 83, 90-2.
Bernstein, R., Bagg, A., Pinto, M., Lewis; D. and Mendelow, B. (1986). Chromosome 3q21 abnormalities associated with hyperactive thrombopoiesis in acute blastic transformation of chronic myeloid leukemia. Blood 68, 652-7.
Bray, P., Lichter, P., Thiesen, H. J., Ward, D. C. and Dawid, I. B. (1991). Characterization and mapping of human genes encoding zinc finger proteins. Proc Natl Acad Sci USA 88, 9563-7.
Burns, C. E., DeBlasio, T., Zhou, Y., Zhang, J., Zon, L. and Nimer, S. D. (2002). Isolation and characterization of runxa and runxb, zebrafish members of the runt family of transcriptional regulators. Exp Hematol 30, 1381-9.
Choi, K., Kennedy, M., Kazarov, A., Papadimitriou, J. C. and Keller, G. (1998). A common precursor for hematopoietic and endothelial cells. Development 125, 725-32.
Chung, Y. S., Zhang, W. J., Arentson, E., Kingsley, P. D., Palis, J. and Choi, K. (2002). Lineage analysis of the hemangioblast as defined by FLK1 and SCL expression. Development 129, 5511-20.
Cross, L. M., Cook, M. A., Lin, S., Chen, J. N. and Rubinstein, A. L. (2003). Rapid analysis of angiogenesis drugs in a live fluorescent zebrafish assay. Arterioscler Thromb Vasc Biol 23, 911-2.
D'Souza, S. L., Elefanty, A. G. and Keller, G. (2005). SCL/Tal-1 is essential for hematopoietic commitment of the hemangioblast but not for its development. Blood 105, 3862-70.
Davidson, A. J., Ernst, P., Wang, Y., Dekens, M. P., Kingsley, P. D., Palis, J., Korsmeyer, S. J., Daley, G. Q. and Zon, L. I. (2003). cdx4 mutants fail to specify blood progenitors and can be rescued by multiple hox genes. Nature 425, 300-6.
Fehling, H. J., Lacaud, G., Kubo, A., Kennedy, M., Robertson, S., Keller, G. and Kouskoff, V. (2003). Tracking mesoderm induction and its specification to the hemangioblast during embryonic stem cell differentiation. Development 130, 4217-27.
Feraud, O., Cao, Y. and Vittet, D. (2001). Embryonic stem cell-derived embryoid bodies development in collagen gels recapitulates sprouting angiogenesis. Lab Invest 81, 1669-81.
Fouquet, B., Weinstein, B. M., Serluca, F. C. and Fishman, M. C. (1997). Vessel patterning in the embryo of the zebrafish: guidance by notochord. Dev Biol 183, 37-48.
Gering, M., Rodaway, A. R, Gottgens, B., Patient, R. K. and Green, A. R. (1998). The SCL gene specifies haemangioblast development from early mesoderm. Embo J 17, 4029-45.
Gering, M., Yamada, Y., Rabbitts, T. H. and Patient, R. K. (2003). Lmo2 and Scl/Tal1 convert non-axial mesoderm into haemangioblasts which differentiate into endothelial cells in the absence of Gata1. Development 130, 6187-99.
Grutz, G. G., Bucher, K., Lavenir, I., Larson, T., Larson, R. and Rabbitts, T. H. (1998). The oncogenic T cell LIM-protein Lmo2 forms part of a DNA-binding complex specifically in immature T cells. Embo J 17, 4594-605;
Hall, M. A., Curtis, D. J., Metcalf, D., Elefanty, A. G., Sourris, K., Robb, L., Gothert, J. R., Jane, S. M. and Begley, C. G. (2003). The critical regulator of embryonic hematopoiesis, SCL, is vital in the adult for megakaryopoiesis, erythropoiesis, and lineage choice in CFU-S12. Proc Natl Acad Sci USA 100, 992-7.
Hasegawa, T., Takeuchi, A., Miyaishi, O., Isobe, K. and de Crombrugghe, B. (1997). Cloning and characterization of a transcription factor that binds to the proximal promoters of the two mouse type I collagen genes. J Biol Chem 272, 4915-23.
Herbomel, P., Thisse, B. and Thisse, C. (1999). Ontogeny and behaviour of early macrophages in the zebrafish embryo. Development 126, 3735-45.
Huber, T. L., Kouskoff, V., Fehling, H. J., Palis, J. and Keller, G. (2004). Haemangioblast commitment is initiated in the primitive streak of the mouse embryo. Nature 432, 625-30.
Kaczynski, J., Cook, T. and Urrutia, R. (2003). Sp 1- and Kruppel-like transcription factors. Genome Biol 4, 206.
Kalev-Zylinska, M. L., Horsfield, J. A., Flores, M. V., Postlethwait, J. H., Vitas, M. R., Baas, A. M., Crosier, P. S, and Crosier, K. E. (2002). Runx 1 is required for zebrafish blood and vessel development and expression of a human RUNX1-CBF₂T1 transgene advances a model for studies of leukemogenesis. Development 129, 2015-30.
Kennedy, M., Firpo, M., Choi, K., Wall, C., Robertson, S., Kabrun, N. and Keller, G. (1997). A common precursor for primitive erythropoiesis and definitive haematopoiesis. Nature 386, 488-93.
Lacaud, G., Gore, L., Kennedy, M., Kouskoff, V., Kingsley, P., Hogan, C., Carlsson, L., Speck, N., Palis, J. and Keller, G. (2002). Runx1 is essential for hematopoietic commitment at the hemangioblast stage of development in vitro. Blood 100, 458-66.
Lahlil, R., Lecuyer, E., Herblot, S, and Hoang, T. (2004). SCL assembles a multifactorial complex that determines glycophorin A expression. Mol Cell. Biol 24, 1439-52.
Lecuyer, E., Herblot, S., Saint-Denis, M., Martin, R., Begley, C. G., Porcher, C., Orkin, S. H. and Hoang, T. (2002). The SCL complex regulates c-kit expression in hematopoietic cells through functional interaction with Sp1. Blood 100, 2430-40.
Liao, E. C., Paw, B. H., Oates, A. C., Pratt, S. J., Postlethwait, J. H. and Zon, L. I. (1998). SCL/Tal-1 transcription factor acts downstream of cloche to specify hematopoietic and vascular progenitors in zebrafish. Genes Dev 12, 621-6.
Liao, W., Bisgrove, B. W., Sawyer, H., Hug, B., Bell, B., Peters, K., Grunwald, D. J. and Stainier, D. Y. (1997). The zebrafish gene cloche acts upstream of a flk-1 homologue to regulate endothelial cell differentiation. Development 124, 381-9.
Long, Q., Meng, A., Wang, H., Jessen, J. R., Farrell, M. J. and Lin, S. (1997). GATA-1 expression pattern can be recapitulated in living transgenic zebrafish using GFP reporter gene. Development 124, 4105-11.
Merchant, J. L., Iyer, G. R., Taylor, B. R., Kitchen, J. R., Mortensen, E. R., Wang, Z., Flintoft, R. J., Michel, J. B. and Bassel-Duby, R. (1996). ZBP-89, a Kruppel-like zinc finger protein, inhibits epidermal growth factor induction of the gastrin promoter. Mol Cell Biol 16, 6644-53.
Mikkola, H. K., Klintman, J., Yang, H., Hock, H., Schlaeger, T. M., Fujiwara, Y. and Orkin, S. H. (2003). Haematopoietic stem cells retain long-term repopulating activity and multipotency in the absence of stem-cell leukaemia SCL/tal-1 gene. Nature 421, 547-51.
Mucenski, M. L., McLain, K., Kier, A. B., Swerdlow, S. H., Schreiner, C. M., Miller, T. A., Pietryga, D. W., Scott, W. J., Jr. and Potter, S. S. (1991). A functional c-myb gene is required for normal murine fetal hepatic hematopoiesis. Cell 65, 677-89;
Okuda, T., van Deursen, J., Hiebert, S. W., Grosveld, G. and Downing, J. R. (1996). AML1, the target of multiple chromosomal translocations in human leukemia, is essential for normal fetal liver hematopoiesis. Cell 84, 321-30.
Orkin, S. H. and Zon, L. I. (2002). Hematopoiesis and stem cells: plasticity versus developmental heterogeneity. Nat Immunol 3, 323-8.
Palis, J., Robertson, S., Kennedy, M., Wall, C. and Keller, G. (1999). Development of erythroid and myeloid progenitors in the yolk sac and embryo proper of the mouse. Development 126, 5073-84.
Park, C., Ma, Y. D. and Choi, K. (2005). Evidence for the hemangioblast. Exp Hematol 33, 965-70.
Park, H., Shelley, C. S. and Arnaout, M. A. (2003). The zinc finger transcription factor ZBP-89 is a repressor of the human beta 2-integrin CD11 b gene. Blood 101, 894-902.
Passantino, R., Antona, V., Barbieri, G., Rubino, P., Melchionna, R., Cossu, G., Feo, S, and Giallongo, A. (1998). Negative regulation of beta enolase gene transcription in embryonic muscle is dependent upon a zinc finger factor that binds to the G-rich box within the muscle-specific enhancer. J Biol Chem 273, 484-94.
Patterson, L. J., Gering, M. and Patient, R. (2005). Scl is required for dorsal aorta as well as blood formation in zebrafish embryos. Blood 105, 3502-11.
Pekarsky, Y., Zabarovsky, E., Kashuba, V., Drabkin, H., Sandberg, A. A., Morgan, R., Rynditch, A. and Gardiner, K. (1995). Cloning of breakpoints in 3q21 associated with hematologic malignancy. Cancer Genet Cytogenet. 80, 1-8.
Porcher, C., Liao, E. C., Fujiwara, Y., Zon, L. I. and Orkin, S. H. (1999). Specification of hematopoietic and vascular development by the bHLH transcription factor SCL without direct DNA binding. Development 126, 4603-15.
Porcher, C., Swat, W., Rockwell, K., Fujiwara, Y., Alt, F. W. and Orkin, S. H. (1996). The T cell leukemia oncoprotein SCL/tal-1 is essential for development of all hematopoietic lineages. Cell 86, 47-57.
Ransom, D. G., Bahary, N., Niss, K., Traver, D., Burns, C., Trede, N. S., Paffett-Lugassy, N., Saganic, W. J., Lim, C. A., Hersey, C. et al. (2004). The zebrafish moonshine gene encodes transcriptional intermediary factor 1 gamma, an essential regulator of hematopoiesis. PLoS Biol 2, E237.
Robb, L. and Begley, C. G. (1997). The SCL/TAL1 gene: roles in normal and malignant haematopoiesis. Bioessays 19, 607-13.
Robb, L., Elwood, N. J., Elefanty, A. G., Kontgen, F., Li, R., Barnett, L. D. and Begley, C. G. (1996). The scl gene product is required for the generation of all hematopoietic lineages in the adult mouse. Embo J 15, 4123-9.
Rodriguez, P., Bonte, E., Krijgsveld, J., Kolodziej, K. E., Guyot, B., Heck, A. J., Vyas, P., de Boer, E., Grosveld, F. and Strouboulis, L (2005). GATA-1 forms distinct activating and repressive complexes in erythroid cells. Embo J 24, 2354-66.
Sanchez, M. J., Bockamp, E. O., Miller, J., Gambardella, L. and Green, A. R. (2001). Selective rescue of early haematopoietic progenitors in Scl(−/−) mice by expressing Scl under the control of a stem cell enhancer. Development 128, 4815-27.
Shivdasani, R. A., Mayer, E. L. and Orkin, S. H. (1995). Absence of blood formation in mice lacking the T-cell leukaemia oncoprotein tal-1/SCL. Nature 373, 432-4.
Stainier, D. Y., Weinstein, B. M., Detrich, H. W., 3rd, Zon, L. I. and Fislunan, M. C. (1995). Cloche, an early acting zebrafish gene, is required by both the endothelial and hematopoietic lineages. Development 121, 3141-50.
Takeuchi, A., Mishina, Y., Miyaishi, O., Kojima, E., Hasegawa, T. and Isobe, K. (2003). Heterozygosity with respect to Zfp148 causes complete loss of fetal germ cells during mouse embryogenesis. Nat Genet. 33, 172-6.
Tenen, D. G., Hromas, R., Licht, J. D. and Zhang, D. E. (1997). Transcription factors, normal myeloid development, and leukemia. Blood 90, 489-519.
Thompson, M. A., Ransom, D. G., Pratt, S. J., MacLennan, H., Kieran, M. W., Detrich, H. W., 3rd, Vail, B., Huber, T. L., Paw, B., Brownlie, A. J. et al. (1998). The cloche and spadetail genes differentially affect hematopoiesis and vasculogenesis. Dev Biol 197, 248-69.
Tsai, F. Y., Keller, G., Kuo, F. C., Weiss, M., Chen, J., Rosenblatt, M., Alt, F. W. and Orkin, S. H. (1994). An early haematopoietic defect in mice lacking the transcription factor GATA-2. Nature 371, 221-6.
Visvader, J. E., Fujiwara, Y. and Orkin, S. H. (1998). Unsuspected role for the T-cell leukemia protein SCL/tal-1 in vascular development. Genes Dev 12, 473-9.
Wadman, L A., Osada, H., Grutz, G. G., Agulnick, A. D., Westphal, H., Forster, A. and Rabbitts, T. H. (1997). The LAM-only protein Lmo2 is a bridging molecule assembling an erythroid, DNA-binding complex which includes the TAL1, E47, GATA-1 and Ldbl/NLI proteins. Embo J 16, 3145-57.
Wang, Z., Cohen, K., Shao, Y., Mole, P., Dombkowski, D. and Scadden, D. T. (2004). Ephrin receptor, EphB4, regulates ES cell differentiation of primitive mammalian hemangioblasts, blood, cardiomyocytes, and blood vessels. Blood 103, 100-9.
Weinstein, B. M., Schier, A. F., Abdelilah, S., Malicki, J., Solnica-Krezel, L., Stemple, D. L., Stainier, D. Y., Zwartkruis, F., Driever, W. and Fishman, M. C. (1996). Hematopoietic mutations in the zebrafish. Development 123, 303-9.
Westerfield, M. (1993). The Zebrafish Book: A guide for the Laboratory use of Zebrafish(Brachydanio redio). Eugene, Oreg.: University of Oregon Press.
Willey, S., Ayuso-Sacido, A., Zhang, H., Fraser, S. T., Sahr, K. E., Adlam, M. J., Kyba, M., Daley, G. Q., Keller, G. and Baron, M. H. (2006). Acceleration of mesoderm development and expansion of hematopoietic progenitors in differentiating ES cells by the mouse Mix-like homeodomain transcription factor. Blood 107, 3122-30.
Yamagata, N., Shimazald, C., Kikuta, T., Hirai, H., Sumikuma, T., Sudo, Y., Ashihara, E., Goto, H., Inaba, T., Fujita, N. et al. (1997). A translocation between 3q21 and 12q24 in a patient with minimally differentiated acute myeloid leukemia (AML-M0). Cancer Genet Cytogenet 97, 90-3.

1. A method for modulating hemangioblast cell fate, the method comprising contacting a hemangioblast with an agent that alters expression of a ZBP-89 polypeptide or nucleic acid molecule in said hemangioblast, thereby modulating hemangioblast cell fate.

2. The method of claim 1, wherein the method increases or decreases ZBP-89 expression.

3. The method of claim 2, wherein the method alters expression of Scl, Lmo2, or Gata-1 expression in the cell.

4. The method of claim 1, wherein the cell assumes a hematopoietic or endothelial cell fate.

5. The method of claim 2, wherein the method alters expression of the endothelial markers flk1 and tie1.

6. (canceled)

7. A method for altering angiogenesis in a cell, tissue or organ, the method comprising contacting a cell, tissue, or organ with a ZBP-89 polypeptide, a nucleic acid encoding said polypeptide, or a ZBP-89 inhibitory nucleic acid molecule, thereby altering angiogenesis.

8. The method of claim 7, wherein the ZBP-89 polypeptide has at least about 85%, 90%, 95% or 99% sequence identity to a human ZBP-89 polypeptide.

9. The method of claim 7, wherein the contacting occurs in vivo or in vitro.

10. The method of claim 7, wherein the cell, hematopoietic stem cell, tissue, or organ is present in a subject.

11-15. (canceled)

16. The method of claim 7, wherein the contacting increases or decreases expression of SCL, Lmo2, or Gata-1.

17. The method of claim 1, wherein the contacting increases or decreases expression of endothelial marker Flk1 or Tie1.

18. The method of claim 1, wherein the ZBP-89 polypeptide is administered to the subject by an oral, intravenous, or transdermal route.

19. The method of claim 1, wherein the ZBP-89 polypeptide is no longer expressed in the cells at the time of administering the cells to the subject.

20. The method of claim 1, wherein the method further comprises administering the cells to a subject.

21. The method of claim 20, wherein the cells are administered via an intravenous route.

22-53. (canceled)

54. The method of claim 7, wherein the contacting increases or decreases expression of endothelial marker Flk1 or Tie1.

55. The method of claim 7, wherein the ZBP-89 polypeptide is administered to the subject by an oral, intravenous, or transdermal route.

56. The method of claim 7, wherein the ZBP-89 polypeptide is no longer expressed in the cells at the time of administering the cells to the subject.

57. The method of claim 7, wherein the method further comprises administering the cells to a subject.

58. The method of claim 57, wherein the cells are administered via an intravenous route.


SEQUENCE LISTING (21 sequences)

SEQ ID NO: 1 - DNA, 21 bp, Artificial Sequence
  Description of Artificial Sequence Synthetic primer
gagatttccttcagcgtttac

SEQ ID NO: 2 - DNA, 20 bp, Artificial Sequence
  Description of Artificial Sequence Synthetic primer
tttggaagggtctggttgtc

SEQ ID NO: 3 - DNA, 24 bp, Artificial Sequence
  Description of Artificial Sequence Synthetic primer
tgctggaggacatgaatccaccag

SEQ ID NO: 4 - DNA, 24 bp, Artificial Sequence
  Description of Artificial Sequence Synthetic primer
tggagagagactctgggactgctc

SEQ ID NO: 5 - DNA, 32 bp, Artificial Sequence
  Description of Artificial Sequence Synthetic primer
aagagaattcatgaacattgatgacaagctgg

SEQ ID NO: 6 - DNA, 18 bp, Artificial Sequence
  Description of Artificial Sequence Synthetic primer
ctgctctagagcctgctg

SEQ ID NO: 7 - DNA, 25 bp, Artificial Sequence
  Description of Artificial Sequence Synthetic primer
tccccacctggcagcaggcatcttg

SEQ ID NO: 8 - DNA, 24 bp, Artificial Sequence
  Description of Artificial Sequence Synthetic primer
agcttttgttcagccaaaggtttg

SEQ ID NO: 9 - DNA, 18 bp, Artificial Sequence
  Description of Artificial Sequence Synthetic primer
cagcaggctctagagcag

SEQ ID NO: 10 - DNA, 30 bp, Artificial Sequence
  Description of Artificial Sequence Synthetic primer
aagagcggccgctcagccaaaggtttggct

SEQ ID NO: 11 - DNA, 25 bp, Artificial Sequence
  Description of Artificial Sequence Synthetic oligonucleotide
cctccagcttgtcatcaatgttcat

SEQ ID NO: 12 - DNA, 25 bp, Artificial Sequence
  Description of Artificial Sequence Synthetic oligonucleotide
gtcaaaatattacctgatggcaata

SEQ ID NO: 13 - DNA, 25 bp, Artificial Sequence
  Description of Artificial Sequence Synthetic oligonucleotide
ccacctgcttgtgatcaatcttgat

SEQ ID NO: 14 - DNA, 23 bp, Artificial Sequence
  Description of Artificial Sequence Synthetic primer
gaaaagcctttccagtgcaatca

SEQ ID NO: 15 - DNA, 23 bp, Artificial Sequence
  Description of Artificial Sequence Synthetic primer
atctttgacagctgtttctgcac

SEQ ID NO: 16 - DNA, 21 bp, Artificial Sequence
  Description of Artificial Sequence Synthetic primer
cggcatagacgaaatgcagtc

SEQ ID NO: 17 - DNA, 21 bp, Artificial Sequence
  Description of Artificial Sequence Synthetic primer
cctggtgaggcaaacttcgat

SEQ ID NO: 18 - DNA, 20 bp, Artificial Sequence
  Description of Artificial Sequence Synthetic primer
tgaccacagtccatgccatc

SEQ ID NO: 19 - DNA, 21 bp, Artificial Sequence
  Description of Artificial Sequence Synthetic primer
gacggacacattgggggttag

SEQ ID NO: 20 - protein, 794 aa, Homo sapiens
MNIDDKLEGLFLKCGGIDEMQSSRTMVVMGGVSGQSTVSGELQDSVLQDRSMPHQEILAADEVLQESEMRQQDMISHDELMVHEETVKNDEEQMETHERLPQGLQYALNVPISVKQEITFTDVSEQLMRDKKQIREPVDLQKKKKRKQRSPAKILTINEDGSLGLKTPKSHVCEHCNAAFRTNYHLQRHVFIHTGEKPFQCSQCDMRFIQKYLLQRHEKIHTGEKPFRCDECGMRFIQKYHMERHKRTHSGEKPYQCEYCLQYFSRTDRVLKHKRMCHENHDKKLNTCAIKGGLLTSEEDSGFSTSPKDNSLPKKKRQKTEKKSSGMDKESALDKSDLKKDKNDYLPVYSSSTKVKDEYMVAEYAVEMPHSSVGGSHLEDASGEIHPPKLVLKKINSKRSLKQPLEQNQTISPLSTYEESKVSKYAFELVDKQALLDSEGNADIDQVDNLQEGPSKPVHSSTNYDDAMQFLKKKRYLQAASNNSREYALNVGTIRSQPSVTQAAVASVIDESTTASILESQALNVEIKSNHDKNVIPDEVLQTLLDHYSHKANGQHEISFSVADTEVTSSISINSSEVPEVTPSENVGSSSQASSSDKANMLQEYSKFLQQALDRTSQNDAYLNSPSLNFVTDNQTLPNQPAFSSIDKQVYATMPINSFRSGMNSPLRTTPDKSHFGLIVGDSQHSFPFSGDETNHASATSTQDFLDQVTSQKKAEAQPVHQAYQMSSFEQPFRAPYHGSRAGIATQFSTANGQVNLRGPGTSAEFSEFPLVNVNDNRAGMTSSPDATTGQTFG

SEQ ID NO: 21 - protein, 808 aa, Danio rerio
MNIDDKLEGMLLKYSPVGLQHPSRGLGPSRVDGRGRRGSLDMTLGERSLANHPLLAEDDDDEDEEEDDDDDLAGEGLTSHDLISQDDLMVHEETVKNDGQDEAAFSHRISNKLHYTLSMPVNIKQEMKVPDSLILNKKEKKPGRDPSDCHKKKKRKQRSPAKILTINEDGSLGHQNPKSHICEHCNAAFRTNYHLQRHVFIHTGEKPFQCNQCDMRFIQKYLLQRHEKIHTGEKPFRCDECGMKFIQKYHMERHKRTHSGEKPYQCDYCHQFFSRTDRVLKHRRMCHENKDRKVQKTAVKDSPLRTPENLGFSFPAKDCTLPKKKRQKTSGKSRASITNPAVDKVVEADMDEKTEDRLAKSECLPLYAVATKVKDEYVVTDYSVELPDSPPGSRHLAEEESNDEIISPPKLVLKKIASRRGFKQQALEQSQSLSPLSSFEESKVTRYTFEIVDNKGLLDVETNPDMESVEALQVGQAKPTGSSTNYDDAMQFVKKRRYLQAATANTSRDYALNVSSIVSQSSVTQASVATVIDETVPATILSETQTLNVEIKSSNEKNVLPDEVLQTLLDHYSNKANGQAEISFSVADTEVTSSISINSSDESSPAEALVTSSQAPPTEKASLLQEYSKFLQQALERTSQNDTYLNNQSLTFVNDSASLAGQPLFSTEKQFTSPPRFRPTMNSPLRSTLEKPNFNLLVDTQHSFSFSGDETNPSSVSPTEDFLDQVTSPKKTDPQSISQTFQITTFDQNFRSQFPSSRSGISPQFSIASGQVTLRGHGGADFPEFSLVNETRTQLTSSPDATSSQTFG